SEQUENCE LISTING (282 sequences)

SEQ ID NO: 1 - DNA, 20 bp, Artificial Sequence
  Antisense oligonucleotide
cagcaattggtgtattcaaa

SEQ ID NO: 2 - DNA, 20 bp, Artificial Sequence
  Antisense oligonucleotide
ctcttcctcgagccgcccca

SEQ ID NO: 3 - DNA, 20 bp, Artificial Sequence
  Antisense oligonucleotide
ttctgaggcactggtgtctt

SEQ ID NO: 4 - DNA, 20 bp, Artificial Sequence
  Antisense oligonucleotide
tcaccgtctgaccgagcagt

SEQ ID NO: 5 - DNA, 20 bp, Artificial Sequence
  Antisense oligonucleotide
tgtgaatgcgtggctcgcta

SEQ ID NO: 6 - DNA, 20 bp, Artificial Sequence
  Antisense oligonucleotide
tctgtgagcagcagtggccc

SEQ ID NO: 7 - DNA, 20 bp, Artificial Sequence
  Antisense oligonucleotide
ggcatcatcctcactgcuuc

SEQ ID NO: 8 - DNA, 20 bp, Artificial Sequence
  Antisense oligonucleotide
ggcagacatcatcctggcau

SEQ ID NO: 9 - DNA, 20 bp, Artificial Sequence
  Antisense oligonucleotide
ggcgcgaggcagacatcatc

SEQ ID NO: 10 - DNA, 20 bp, Artificial Sequence
  Antisense oligonucleotide
tgggatcagagtcccagcca

SEQ ID NO: 11 - DNA, 20 bp, Artificial Sequence
  Antisense oligonucleotide
caggagaggaaggccatggc

SEQ ID NO: 12 - DNA, 20 bp, Artificial Sequence
  Antisense oligonucleotide
cccagctttctggtctcacg

SEQ ID NO: 13 - DNA, 20 bp, Artificial Sequence
  Antisense oligonucleotide
aaccacctccacgcagggct

SEQ ID NO: 14 - DNA, 20 bp, Artificial Sequence
  Antisense oligonucleotide
cattgataagtaatattagg

SEQ ID NO: 15 - DNA, 20 bp, Artificial Sequence
  Antisense oligonucleotide
tgtagaaattcagctccatg

SEQ ID NO: 16 - DNA, 20 bp, Artificial Sequence
  Antisense oligonucleotide
ggggaggttgtcggggattt

SEQ ID NO: 17 - DNA, 20 bp, Artificial Sequence
  Antisense oligonucleotide
cucaggtccaggttctuggu

SEQ ID NO: 18 - DNA, 20 bp, Artificial Sequence
  Antisense oligonucleotide
tcaggggattaaagctcagg

SEQ ID NO: 19 - DNA, 20 bp, Artificial Sequence
  Antisense oligonucleotide
gctatagctgcctaaatgcc

SEQ ID NO: 20 - DNA, 20 bp, Artificial Sequence
  Antisense oligonucleotide
agttctgggaaactgaagaa

SEQ ID NO: 21 - DNA, 20 bp, Artificial Sequence
  Antisense oligonucleotide
tggataaatccagcacctgc

SEQ ID NO: 22 - DNA, 20 bp, Artificial Sequence
  Antisense oligonucleotide
aattgtctggatttcacacc

SEQ ID NO: 23 - DNA, 20 bp, Artificial Sequence
  Antisense oligonucleotide
ctctgatatgccccatcttc

SEQ ID NO: 24 - DNA, 20 bp, Artificial Sequence
  Antisense oligonucleotide
ggcutaggctctgataugcc

SEQ ID NO: 25 - DNA, 20 bp, Artificial Sequence
  Antisense oligonucleotide
aggtagagaggtggcttagg

SEQ ID NO: 26 - DNA, 20 bp, Artificial Sequence
  Antisense oligonucleotide
ggggtttcctgtcaatatta

SEQ ID NO: 27 - DNA, 20 bp, Artificial Sequence
  Antisense oligonucleotide
cccagggctaaactctggat

SEQ ID NO: 28 - DNA, 20 bp, Artificial Sequence
  Antisense oligonucleotide
ttgatagtccagaaaaggct

SEQ ID NO: 29 - DNA, 20 bp, Artificial Sequence
  Antisense oligonucleotide
agccaccagcttctgtaaac

SEQ ID NO: 30 - DNA, 20 bp, Artificial Sequence
  Antisense oligonucleotide
ucuccacagccaccagcuuc

SEQ ID NO: 31 - DNA, 20 bp, Artificial Sequence
  Antisense oligonucleotide
gatgctagatttgtctccac

SEQ ID NO: 32 - DNA, 20 bp, Artificial Sequence
  Antisense oligonucleotide
caatggggaagttctctaga

SEQ ID NO: 33 - DNA, 20 bp, Artificial Sequence
  Antisense oligonucleotide
tttcaaagttttgagatgtc

SEQ ID NO: 34 - DNA, 20 bp, Artificial Sequence
  Antisense oligonucleotide
ttgtgagccacattaagttc

SEQ ID NO: 35 - DNA, 20 bp, Artificial Sequence
  Antisense oligonucleotide
atttgaaagattggataaga

SEQ ID NO: 36 - DNA, 20 bp, Artificial Sequence
  Antisense oligonucleotide
attagaaaaatactcaggta

SEQ ID NO: 37 - DNA, 20 bp, Artificial Sequence
  Antisense oligonucleotide
aagtgctctagattggtcag

SEQ ID NO: 38 - DNA, 20 bp, Artificial Sequence
  Antisense oligonucleotide
tcttgttgctggaaaggtcc

SEQ ID NO: 39 - DNA, 20 bp, Artificial Sequence
  Antisense oligonucleotide
tgtgcaataaatactttgaa

SEQ ID NO: 40 - DNA, 20 bp, Artificial Sequence
  Antisense oligonucleotide
tgatgtagaacccgcaagtc

SEQ ID NO: 41 - DNA, 20 bp, Artificial Sequence
  Antisense oligonucleotide
agagattgagtaggggcatt

SEQ ID NO: 42 - DNA, 20 bp, Artificial Sequence
  Antisense oligonucleotide
gttcagggacaggtctaaag

SEQ ID NO: 43 - DNA, 20 bp, Artificial Sequence
  Antisense oligonucleotide
ggttggataaagttcatagg

SEQ ID NO: 44 - DNA, 20 bp, Artificial Sequence
  Antisense oligonucleotide
taatttctttaaatgcacct

SEQ ID NO: 45 - DNA, 20 bp, Artificial Sequence
  Antisense oligonucleotide
taaagtcagcttatgaagcc

SEQ ID NO: 46 - DNA, 20 bp, Artificial Sequence
  Antisense oligonucleotide
aaactatcaaaattatttct

SEQ ID NO: 47 - DNA, 20 bp, Artificial Sequence
  Antisense oligonucleotide
tacaagttttcattacattt

SEQ ID NO: 48 - DNA, 20 bp, Artificial Sequence
  Antisense oligonucleotide
taaaccagccagaccttgaa

SEQ ID NO: 49 - DNA, 20 bp, Artificial Sequence
  Antisense oligonucleotide
gauggacttctaaaccagcc

SEQ ID NO: 50 - DNA, 20 bp, Artificial Sequence
  Antisense oligonucleotide
agaaccaaacgatggacttc

SEQ ID NO: 51 - DNA, 20 bp, Artificial Sequence
  Antisense oligonucleotide
cttcatttctaaattctccc

SEQ ID NO: 52 - DNA, 20 bp, Artificial Sequence
  Antisense oligonucleotide
gtcaaacttttccaagtttc

SEQ ID NO: 53 - DNA, 20 bp, Artificial Sequence
  Antisense oligonucleotide
aggccctctagagcagattt

SEQ ID NO: 54 - DNA, 20 bp, Artificial Sequence
  Antisense oligonucleotide
cttcaatggtcaaattgcac

SEQ ID NO: 55 - DNA, 20 bp, Artificial Sequence
  Antisense oligonucleotide
taagtatgctaatcggaatt

SEQ ID NO: 56 - DNA, 20 bp, Artificial Sequence
  Antisense oligonucleotide
atatcatcgaggtagtagtc

SEQ ID NO: 57 - DNA, 20 bp, Artificial Sequence
  Antisense oligonucleotide
aacaattaaataagtcaata

SEQ ID NO: 58 - DNA, 20 bp, Artificial Sequence
  Antisense oligonucleotide
aaatgaagaaacatttgtca

SEQ ID NO: 59 - DNA, 20 bp, Artificial Sequence
  Antisense oligonucleotide
atagtcacactcaccaggga

SEQ ID NO: 60 - DNA, 20 bp, Artificial Sequence
  Antisense oligonucleotide
aaaagtcttttaccctttca

SEQ ID NO: 61 - DNA, 20 bp, Artificial Sequence
  Antisense oligonucleotide
ttgccatccgaaattataag

SEQ ID NO: 62 - DNA, 20 bp, Artificial Sequence
  Antisense oligonucleotide
cagttaactaattctaaatg

SEQ ID NO: 63 - DNA, 20 bp, Artificial Sequence
  Antisense oligonucleotide
tgggaaactgtccaaattta

SEQ ID NO: 64 - DNA, 20 bp, Artificial Sequence
  Antisense oligonucleotide
gagagatttgagtttcaatg

SEQ ID NO: 65 - DNA, 20 bp, Artificial Sequence
  Antisense oligonucleotide
gaagtgaaagtaagcctttt

SEQ ID NO: 66 - DNA, 20 bp, Artificial Sequence
  Antisense oligonucleotide
aagcattcccacctttgttg

SEQ ID NO: 67 - DNA, 20 bp, Artificial Sequence
  Antisense oligonucleotide
tggtagatcaacttctgaaa

SEQ ID NO: 68 - DNA, 20 bp, Artificial Sequence
  Antisense oligonucleotide
agatctagaaactcaaggct

SEQ ID NO: 69 - DNA, 20 bp, Artificial Sequence
  Antisense oligonucleotide
aactcaagccatttctactg

SEQ ID NO: 70 - DNA, 20 bp, Artificial Sequence
  Antisense oligonucleotide
ttgagaacagcaacctttga

SEQ ID NO: 71 - DNA, 20 bp, Artificial Sequence
  Antisense oligonucleotide
ctggttgtcccaaaatcact

SEQ ID NO: 72 - DNA, 20 bp, Artificial Sequence
  Antisense oligonucleotide
tcagatctaaatactttagg

SEQ ID NO: 73 - DNA, 20 bp, Artificial Sequence
  Antisense oligonucleotide
ggtaataacaccattgaagc

SEQ ID NO: 74 - DNA, 20 bp, Artificial Sequence
  Antisense oligonucleotide
cccaagaagtttgaactcat

SEQ ID NO: 75 - DNA, 20 bp, Artificial Sequence
  Antisense oligonucleotide
gatgttctagttgttctaag

SEQ ID NO: 76 - DNA, 20 bp, Artificial Sequence
  Antisense oligonucleotide
attggaatgctggaaatcca

SEQ ID NO: 77 - DNA, 20 bp, Artificial Sequence
  Antisense oligonucleotide
aactcactcatttgtttcaa

SEQ ID NO: 78 - DNA, 20 bp, Artificial Sequence
  Antisense oligonucleotide
tgagtgataggaatactgaa

SEQ ID NO: 79 - DNA, 20 bp, Artificial Sequence
  Antisense oligonucleotide
gtcaaggtaaatgaggtttc

SEQ ID NO: 80 - DNA, 20 bp, Artificial Sequence
  Antisense oligonucleotide
ctggtgtgagtatgagaaat

SEQ ID NO: 81 - DNA, 20 bp, Artificial Sequence
  Antisense oligonucleotide
agatgccattgaaagcaact

SEQ ID NO: 82 - DNA, 20 bp, Artificial Sequence
  Antisense oligonucleotide
gagactggacaagccattga

SEQ ID NO: 83 - DNA, 20 bp, Artificial Sequence
  Antisense oligonucleotide
ccagccattttcaagacttc

SEQ ID NO: 84 - DNA, 20 bp, Artificial Sequence
  Antisense oligonucleotide
agttttcctggaaagaattg

SEQ ID NO: 85 - DNA, 20 bp, Artificial Sequence
  Antisense oligonucleotide
tgtgaagatatctggaagga

SEQ ID NO: 86 - DNA, 20 bp, Artificial Sequence
  Antisense oligonucleotide
ccaggaaggtcaagttucuc

SEQ ID NO: 87 - DNA, 20 bp, Artificial Sequence
  Antisense oligonucleotide
gacactgagagaggtccagg

SEQ ID NO: 88 - DNA, 20 bp, Artificial Sequence
  Antisense oligonucleotide
tggagacaactgctccagtt

SEQ ID NO: 89 - DNA, 20 bp, Artificial Sequence
  Antisense oligonucleotide
gagagtgagttaaatgctgt

SEQ ID NO: 90 - DNA, 20 bp, Artificial Sequence
  Antisense oligonucleotide
tatttagtacctgaagactg

SEQ ID NO: 91 - DNA, 20 bp, Artificial Sequence
  Antisense oligonucleotide
aaagaagttgttgtggctca

SEQ ID NO: 92 - DNA, 20 bp, Artificial Sequence
  Antisense oligonucleotide
taaggaaacgtatccaatga

SEQ ID NO: 93 - DNA, 20 bp, Artificial Sequence
  Antisense oligonucleotide
ggagggagttcagacactta

SEQ ID NO: 94 - DNA, 20 bp, Artificial Sequence
  Antisense oligonucleotide
gagactgtaatcaagaacct

SEQ ID NO: 95 - DNA, 20 bp, Artificial Sequence
  Antisense oligonucleotide
ttggaagtcattatgtgatt

SEQ ID NO: 96 - DNA, 20 bp, Artificial Sequence
  Antisense oligonucleotide
aatgctgtagttcctgtttt

SEQ ID NO: 97 - DNA, 20 bp, Artificial Sequence
  Antisense oligonucleotide
gaaagctagactacttggaa

SEQ ID NO: 98 - DNA, 20 bp, Artificial Sequence
  Antisense oligonucleotide
tcattctgagtaagatttaa

SEQ ID NO: 99 - DNA, 20 bp, Artificial Sequence
  Antisense oligonucleotide
gttcacaagtacaagcaaag

SEQ ID NO: 100 - DNA, 20 bp, Artificial Sequence
  Antisense oligonucleotide
ccautgcaggaaactcuggu

SEQ ID NO: 101 - DNA, 20 bp, Artificial Sequence
  Antisense oligonucleotide
tctggtccttgatccattgc

SEQ ID NO: 102 - DNA, 20 bp, Artificial Sequence
  Antisense oligonucleotide
ccaagagctgcctctggucc

SEQ ID NO: 103 - DNA, 20 bp, Artificial Sequence
  Antisense oligonucleotide
ttcgttcaacttccaccaag

SEQ ID NO: 104 - DNA, 20 bp, Artificial Sequence
  Antisense oligonucleotide
ctgaaggtgttgcacattcc

SEQ ID NO: 105 - DNA, 20 bp, Artificial Sequence
  Antisense oligonucleotide
acaggcatgccctgcttatc

SEQ ID NO: 106 - DNA, 20 bp, Artificial Sequence
  Antisense oligonucleotide
aggtgatattcaaactcagc

SEQ ID NO: 107 - DNA, 20 bp, Artificial Sequence
  Antisense oligonucleotide
gatggtcttattcatctgac

SEQ ID NO: 108 - DNA, 20 bp, Artificial Sequence
  Antisense oligonucleotide
ctgaggaccgacacaccaat

SEQ ID NO: 109 - DNA, 20 bp, Artificial Sequence
  Antisense oligonucleotide
caacagatactacaagcaca

SEQ ID NO: 110 - DNA, 20 bp, Artificial Sequence
  Antisense oligonucleotide
cttatagaccagaactgcta

SEQ ID NO: 111 - DNA, 20 bp, Artificial Sequence
  Antisense oligonucleotide
agcatcaggtgaaaatagaa

SEQ ID NO: 112 - DNA, 20 bp, Artificial Sequence
  Antisense oligonucleotide
actttatgcagccagcaaga

SEQ ID NO: 113 - DNA, 20 bp, Artificial Sequence
  Antisense oligonucleotide
gatgttttcacctctaccat

SEQ ID NO: 114 - DNA, 20 bp, Artificial Sequence
  Antisense oligonucleotide
tagataacaaaggcatcata

SEQ ID NO: 115 - DNA, 20 bp, Artificial Sequence
  Antisense oligonucleotide
cagucctcatcctggcuuga

SEQ ID NO: 116 - DNA, 20 bp, Artificial Sequence
  Antisense oligonucleotide
tactagctcattccttaccc

SEQ ID NO: 117 - DNA, 20 bp, Artificial Sequence
  Antisense oligonucleotide
accccttcttctaaattctt

SEQ ID NO: 118 - DNA, 20 bp, Artificial Sequence
  Antisense oligonucleotide
ggcagagctgaaatggaggc

SEQ ID NO: 119 - DNA, 20 bp, Artificial Sequence
  Antisense oligonucleotide
aataaagtctctgtagtgaa

SEQ ID NO: 120 - DNA, 20 bp, Artificial Sequence
  Antisense oligonucleotide
gcagcaatggccacaccggg

SEQ ID NO: 121 - DNA, 20 bp, Artificial Sequence
  Antisense oligonucleotide
ccuucatggatgatgtuggc

SEQ ID NO: 122 - DNA, 20 bp, Artificial Sequence
  Antisense oligonucleotide
cacctttcggcttttatgga

SEQ ID NO: 123 - DNA, 20 bp, Artificial Sequence
  Antisense oligonucleotide
tgctgggacaccacaacaat

SEQ ID NO: 124 - DNA, 20 bp, Artificial Sequence
  Antisense oligonucleotide
accagcggctctggatgaag

SEQ ID NO: 125 - DNA, 20 bp, Artificial Sequence
  Antisense oligonucleotide
aatctcatattcaaagatac

SEQ ID NO: 126 - DNA, 20 bp, Artificial Sequence
  Antisense oligonucleotide
ugcucagaaactgccagguc

SEQ ID NO: 127 - DNA, 20 bp, Artificial Sequence
  Antisense oligonucleotide
tgataccagcacgactgctc

SEQ ID NO: 128 - DNA, 20 bp, Artificial Sequence
  Antisense oligonucleotide
cttctgcaggacaatgaaga

SEQ ID NO: 129 - DNA, 20 bp, Artificial Sequence
  Antisense oligonucleotide
ctgagcagggtcttctccac

SEQ ID NO: 130 - DNA, 20 bp, Artificial Sequence
  Antisense oligonucleotide
ggtacagctccacctgctgc

SEQ ID NO: 131 - DNA, 20 bp, Artificial Sequence
  Antisense oligonucleotide
agtgttcctgctgagaaggc

SEQ ID NO: 132 - DNA, 20 bp, Artificial Sequence
  Antisense oligonucleotide
ctgtcctcccactccaggta

SEQ ID NO: 133 - DNA, 20 bp, Artificial Sequence
  Antisense oligonucleotide
agatgtgccgccccaggaca

SEQ ID NO: 134 - DNA, 20 bp, Artificial Sequence
  Antisense oligonucleotide
ttttctgagtcgtctccaga

SEQ ID NO: 135 - DNA, 20 bp, Artificial Sequence
  Antisense oligonucleotide
gatttaccatccagcagggc

SEQ ID NO: 136 - DNA, 20 bp, Artificial Sequence
  Antisense oligonucleotide
cacugttccttctggauucc

SEQ ID NO: 137 - DNA, 20 bp, Artificial Sequence
  Antisense oligonucleotide
ccaattgcatcctgtaccca

SEQ ID NO: 138 - DNA, 20 bp, Artificial Sequence
  Antisense oligonucleotide
cagatagatgttgcttcctg

SEQ ID NO: 139 - DNA, 20 bp, Artificial Sequence
  Antisense oligonucleotide
aggtttttatttttcctctt

SEQ ID NO: 140 - DNA, 20 bp, Artificial Sequence
  Antisense oligonucleotide
tgggcaagaaatgcctcagg

SEQ ID NO: 141 - DNA, 20 bp, Artificial Sequence
  Antisense oligonucleotide
gaacaagtgttggacccagc

SEQ ID NO: 142 - DNA, 20 bp, Artificial Sequence
  Antisense oligonucleotide
gcatttaatacttattaact

SEQ ID NO: 143 - DNA, 20 bp, Artificial Sequence
  Antisense oligonucleotide
ataaggcctgacatgtggca

SEQ ID NO: 144 - DNA, 20 bp, Artificial Sequence
  Antisense oligonucleotide
tggaattactcacccttagc

SEQ ID NO: 145 - DNA, 20 bp, Artificial Sequence
  Antisense oligonucleotide
cctgcatatctagtgcacca

SEQ ID NO: 146 - DNA, 20 bp, Artificial Sequence
  Antisense oligonucleotide
gcuccttgagattagcagcc

SEQ ID NO: 147 - DNA, 20 bp, Artificial Sequence
  Antisense oligonucleotide
tttattccctctgcactgga

SEQ ID NO: 148 - DNA, 20 bp, Artificial Sequence
  Antisense oligonucleotide
ctctgtattttagtctagca

SEQ ID NO: 149 - DNA, 20 bp, Artificial Sequence
  Antisense oligonucleotide
tgaaatgcccacctggaaga

SEQ ID NO: 150 - DNA, 20 bp, Artificial Sequence
  Antisense oligonucleotide
ggttccttgactgagttggt

SEQ ID NO: 151 - DNA, 20 bp, Artificial Sequence
  Antisense oligonucleotide
aaatgactttctttgtcatg

SEQ ID NO: 152 - DNA, 20 bp, Artificial Sequence
  Antisense oligonucleotide
acttgatgaggtaagagttg

SEQ ID NO: 153 - DNA, 20 bp, Artificial Sequence
  Antisense oligonucleotide
gttttctctgtctttattca

SEQ ID NO: 154 - DNA, 20 bp, Artificial Sequence
  Antisense oligonucleotide
aaagaacaatgtctctttct

SEQ ID NO: 155 - DNA, 20 bp, Artificial Sequence
  Antisense oligonucleotide
tccattcaaaagactcagga

SEQ ID NO: 156 - DNA, 20 bp, Artificial Sequence
  Antisense oligonucleotide
ggctataacataatacaatt

SEQ ID NO: 157 - DNA, 20 bp, Artificial Sequence
  Antisense oligonucleotide
taccaaaatggttttatgat

SEQ ID NO: 158 - DNA, 20 bp, Artificial Sequence
  Antisense oligonucleotide
acacccagttcagtcaaaac

SEQ ID NO: 159 - DNA, 20 bp, Artificial Sequence
  Antisense oligonucleotide
aatcaaaaaggaaaaagtga

SEQ ID NO: 160 - DNA, 20 bp, Artificial Sequence
  Antisense oligonucleotide
gtagaatttaaattgtattc

SEQ ID NO: 161 - DNA, 20 bp, Artificial Sequence
  Antisense oligonucleotide
ttgacgactgcagtcatcaa

SEQ ID NO: 162 - DNA, 20 bp, Artificial Sequence
  Antisense oligonucleotide
catcttgcatcaggagcccc

SEQ ID NO: 163 - DNA, 20 bp, Artificial Sequence
  Antisense oligonucleotide
cagacttaaaatggaagggg

SEQ ID NO: 164 - DNA, 20 bp, Artificial Sequence
  Antisense oligonucleotide
ctttaacctctgtaaggaga

SEQ ID NO: 165 - DNA, 20 bp, Artificial Sequence
  Antisense oligonucleotide
cttaggaattagccactaga

SEQ ID NO: 166 - DNA, 20 bp, Artificial Sequence
  Antisense oligonucleotide
gcatgtgttaatcaggtttc

SEQ ID NO: 167 - DNA, 20 bp, Artificial Sequence
  Antisense oligonucleotide
aatgaccaggatggttgtga

SEQ ID NO: 168 - DNA, 20 bp, Artificial Sequence
  Antisense oligonucleotide
aaaaatagaacatgctcgag

SEQ ID NO: 169 - DNA, 20 bp, Artificial Sequence
  Antisense oligonucleotide
atatcaggggtgattagtta

SEQ ID NO: 170 - DNA, 20 bp, Artificial Sequence
  Antisense oligonucleotide
ggatatataaaaataaaaat

SEQ ID NO: 171 - DNA, 20 bp, Artificial Sequence
  Antisense oligonucleotide
gacgtaaaaaaatgaaaact

SEQ ID NO: 172 - DNA, 20 bp, Artificial Sequence
  Antisense oligonucleotide
tgatattagcttataggcaa

SEQ ID NO: 173 - DNA, 20 bp, Artificial Sequence
  Antisense oligonucleotide
gtcttaaacaaccttattta

SEQ ID NO: 174 - DNA, 20 bp, Artificial Sequence
  Antisense oligonucleotide
aatatggatatttgaagcac

SEQ ID NO: 175 - DNA, 20 bp, Artificial Sequence
  Antisense oligonucleotide
ttccttgaaaaatagtggtt

SEQ ID NO: 176 - DNA, 20 bp, Artificial Sequence
  Antisense oligonucleotide
cagagtgtacttttccatac

SEQ ID NO: 177 - DNA, 20 bp, Artificial Sequence
  Antisense oligonucleotide
gacatcgagtgacaaagtga

SEQ ID NO: 178 - DNA, 20 bp, Artificial Sequence
  Antisense oligonucleotide
gtaggcaataactttggaat

SEQ ID NO: 179 - DNA, 20 bp, Artificial Sequence
  Antisense oligonucleotide
ttcatgacagtcattactta

SEQ ID NO: 180 - DNA, 20 bp, Artificial Sequence
  Antisense oligonucleotide
caaattatttcaatgctgct

SEQ ID NO: 181 - DNA, 20 bp, Artificial Sequence
  Antisense oligonucleotide
aaaagagtgccccctttaaa

SEQ ID NO: 182 - DNA, 20 bp, Artificial Sequence
  Antisense oligonucleotide
cggaaattttcttcccgttt

SEQ ID NO: 183 - DNA, 20 bp, Artificial Sequence
  Antisense oligonucleotide
tccatgataagaccaggaag

SEQ ID NO: 184 - DNA, 20 bp, Artificial Sequence
  Antisense oligonucleotide
cuuccttcctgcctctagcc

SEQ ID NO: 185 - DNA, 20 bp, Artificial Sequence
  Antisense oligonucleotide
tgaggtcatcccacttcctt

SEQ ID NO: 186 - DNA, 20 bp, Artificial Sequence
  Antisense oligonucleotide
atcaagaaaaggtgacctcc

SEQ ID NO: 187 - DNA, 20 bp, Artificial Sequence
  Antisense oligonucleotide
tcagcccatatgtttctgga

SEQ ID NO: 188 - DNA, 20 bp, Artificial Sequence
  Antisense oligonucleotide
atgaggtcaccccgggttta

SEQ ID NO: 189 - DNA, 20 bp, Artificial Sequence
  Antisense oligonucleotide
cttctgctgcaactcatttc

SEQ ID NO: 190 - DNA, 20 bp, Artificial Sequence
  Antisense oligonucleotide
cttgttctgaaaaaaataaa

SEQ ID NO: 191 - DNA, 20 bp, Artificial Sequence
  Antisense oligonucleotide
cagaggtccatcaaacatca

SEQ ID NO: 192 - DNA, 20 bp, Artificial Sequence
  Antisense oligonucleotide
tgtgtctccctaaagagatt

SEQ ID NO: 193 - DNA, 20 bp, Artificial Sequence
  Antisense oligonucleotide
ggggagggatcccagccatc

SEQ ID NO: 194 - DNA, 20 bp, Artificial Sequence
  Antisense oligonucleotide
ctggcagtgagaagggtaca

SEQ ID NO: 195 - DNA, 20 bp, Artificial Sequence
  Antisense oligonucleotide
taccttcacacgtagttctc

SEQ ID NO: 196 - DNA, 20 bp, Artificial Sequence
  Antisense oligonucleotide
tgtatactccctgccttgaa

SEQ ID NO: 197 - DNA, 20 bp, Artificial Sequence
  Antisense oligonucleotide
tgcccaacaggaaacagcaa

SEQ ID NO: 198 - DNA, 20 bp, Artificial Sequence
  Antisense oligonucleotide
aaaatgtggtcaaggagcat

SEQ ID NO: 199 - DNA, 20 bp, Artificial Sequence
  Antisense oligonucleotide
tgataacatccactcttccc

SEQ ID NO: 200 - DNA, 20 bp, Artificial Sequence
  Antisense oligonucleotide
cagacacattgttttctcaa

SEQ ID NO: 201 - DNA, 20 bp, Artificial Sequence
  Antisense oligonucleotide
tataagaaccccattaattc

SEQ ID NO: 202 - DNA, 20 bp, Artificial Sequence
  Antisense oligonucleotide
tcttttctgggaaccttctt

SEQ ID NO: 203 - DNA, 20 bp, Artificial Sequence
  Antisense oligonucleotide
tgaggaggctggatgaacat

SEQ ID NO: 204 - DNA, 20 bp, Artificial Sequence
  Antisense oligonucleotide
tttcttgaatgttctgtttc

SEQ ID NO: 205 - DNA, 20 bp, Artificial Sequence
  Antisense oligonucleotide
gatgacatcctgattgtcct

SEQ ID NO: 206 - DNA, 20 bp, Artificial Sequence
  Antisense oligonucleotide
gtttttattttcatttccct

SEQ ID NO: 207 - DNA, 20 bp, Artificial Sequence
  Antisense oligonucleotide
taaggtgatatctcattgtg

SEQ ID NO: 208 - DNA, 20 bp, Artificial Sequence
  Antisense oligonucleotide
agtagccattctacctggta

SEQ ID NO: 209 - DNA, 20 bp, Artificial Sequence
  Antisense oligonucleotide
atgacacttcatttttttat

SEQ ID NO: 210 - DNA, 20 bp, Artificial Sequence
  Antisense oligonucleotide
ccaatttctctatatccttg

SEQ ID NO: 211 - DNA, 20 bp, Artificial Sequence
  Antisense oligonucleotide
tccagcagtgaagaagggtt

SEQ ID NO: 212 - DNA, 20 bp, Artificial Sequence
  Antisense oligonucleotide
ctacaccattttccattccc

SEQ ID NO: 213 - DNA, 20 bp, Artificial Sequence
  Antisense oligonucleotide
cgtactgtttttcataacgg

SEQ ID NO: 214 - DNA, 20 bp, Artificial Sequence
  Antisense oligonucleotide
tttaatttttgagaaacctc

SEQ ID NO: 215 - DNA, 20 bp, Artificial Sequence
  Antisense oligonucleotide
gatcatatagcagttctatt

SEQ ID NO: 216 - DNA, 20 bp, Artificial Sequence
  Antisense oligonucleotide
atacagaagtgagattgctg

SEQ ID NO: 217 - DNA, 20 bp, Artificial Sequence
  Antisense oligonucleotide
ttcaattattttgggtatat

SEQ ID NO: 218 - DNA, 20 bp, Artificial Sequence
  Antisense oligonucleotide
tattttcttgaaattctgat

SEQ ID NO: 219 - DNA, 20 bp, Artificial Sequence
  Antisense oligonucleotide
aatgaacatgggagtgtaaa

SEQ ID NO: 220 - DNA, 20 bp, Artificial Sequence
  Antisense oligonucleotide
gtgattgtgaagagtgccac

SEQ ID NO: 221 - DNA, 20 bp, Artificial Sequence
  Antisense oligonucleotide
tttccataactttggaaaca

SEQ ID NO: 222 - DNA, 20 bp, Artificial Sequence
  Antisense oligonucleotide
ttcaatggaaatttgggttg

SEQ ID NO: 223 - DNA, 20 bp, Artificial Sequence
  Antisense oligonucleotide
ttttctttgtccatttattt

SEQ ID NO: 224 - DNA, 20 bp, Artificial Sequence
  Antisense oligonucleotide
ccattgtacgtatatgcaca

SEQ ID NO: 225 - DNA, 20 bp, Artificial Sequence
  Antisense oligonucleotide
tttttaggctgaataatatc

SEQ ID NO: 226 - DNA, 20 bp, Artificial Sequence
  Antisense oligonucleotide
aaataacaggattccccctt

SEQ ID NO: 227 - DNA, 20 bp, Artificial Sequence
  Antisense oligonucleotide
gggtttattcatgttgtcat

SEQ ID NO: 228 - DNA, 20 bp, Artificial Sequence
  Antisense oligonucleotide
tacatagcataatggcctcc

SEQ ID NO: 229 - DNA, 20 bp, Artificial Sequence
  Antisense oligonucleotide
tttctgttacttgctcattt

SEQ ID NO: 230 - DNA, 20 bp, Artificial Sequence
  Antisense oligonucleotide
gaaatcaggcagtatttgtc

SEQ ID NO: 231 - DNA, 20 bp, Artificial Sequence
  Antisense oligonucleotide
ttttagaacctcatataaat

SEQ ID NO: 232 - DNA, 20 bp, Artificial Sequence
  Antisense oligonucleotide
tgcttctatgagtttgacta

SEQ ID NO: 233 - DNA, 20 bp, Artificial Sequence
  Antisense oligonucleotide
ggaaccactgttctattctc

SEQ ID NO: 234 - DNA, 20 bp, Artificial Sequence
  Antisense oligonucleotide
tcccttcctccttttcccta

SEQ ID NO: 235 - DNA, 20 bp, Artificial Sequence
  Antisense oligonucleotide
actccctatttcctcatttc

SEQ ID NO: 236 - DNA, 20 bp, Artificial Sequence
  Antisense oligonucleotide
aattttataccaattagaca

SEQ ID NO: 237 - DNA, 20 bp, Artificial Sequence
  Antisense oligonucleotide
taattcatcttgcatactat

SEQ ID NO: 238 - DNA, 20 bp, Artificial Sequence
  Antisense oligonucleotide
atacagctgatctttagagc

SEQ ID NO: 239 - DNA, 20 bp, Artificial Sequence
  Antisense oligonucleotide
ttcattatacgaactctgct

SEQ ID NO: 240 - DNA, 20 bp, Artificial Sequence
  Antisense oligonucleotide
aagtgcataatacagtattg

SEQ ID NO: 241 - DNA, 20 bp, Artificial Sequence
  Antisense oligonucleotide
taccctcttaacaaaatgtt

SEQ ID NO: 242 - DNA, 20 bp, Artificial Sequence
  Antisense oligonucleotide
agaacacttaacatgagagg

SEQ ID NO: 243 - DNA, 20 bp, Artificial Sequence
  Antisense oligonucleotide
ttgtgtatatgtatatggta

SEQ ID NO: 244 - DNA, 20 bp, Artificial Sequence
  Antisense oligonucleotide
catcacctccaaaagcttcc

SEQ ID NO: 245 - DNA, 20 bp, Artificial Sequence
  Antisense oligonucleotide
atcaaggtaataaatatatc

SEQ ID NO: 246 - DNA, 20 bp, Artificial Sequence
  Antisense oligonucleotide
cctgtcaaaccatcaccaca

SEQ ID NO: 247 - DNA, 20 bp, Artificial Sequence
  Antisense oligonucleotide
agtttagacatagtcacata

SEQ ID NO: 248 - DNA, 20 bp, Artificial Sequence
  Antisense oligonucleotide
ttaatgtatacaatttgatg

SEQ ID NO: 249 - DNA, 20 bp, Artificial Sequence
  Antisense oligonucleotide
tattataaaactgcatatat

SEQ ID NO: 250 - DNA, 20 bp, Artificial Sequence
  Antisense oligonucleotide
cttcattcagacataattga

SEQ ID NO: 251 - DNA, 20 bp, Artificial Sequence
  Antisense oligonucleotide
ttgtcttttcttttttatag

SEQ ID NO: 252 - DNA, 20 bp, Artificial Sequence
  Antisense oligonucleotide
gttttgacaactgaattttg

SEQ ID NO: 253 - DNA, 20 bp, Artificial Sequence
  Antisense oligonucleotide
tgactgtggtcatatttcca

SEQ ID NO: 254 - DNA, 20 bp, Artificial Sequence
  Antisense oligonucleotide
cactcagtaacaaacacttc

SEQ ID NO: 255 - DNA, 20 bp, Artificial Sequence
  Antisense oligonucleotide
aaaccaaacacactctgaaa

SEQ ID NO: 256 - DNA, 20 bp, Artificial Sequence
  Antisense oligonucleotide
gacctgctcaaaccaaacac

SEQ ID NO: 257 - DNA, 20 bp, Artificial Sequence
  Antisense oligonucleotide
caatcaccctagacctgctc

SEQ ID NO: 258 - DNA, 20 bp, Artificial Sequence
  Antisense oligonucleotide
gaaacacacccagggatgtt

SEQ ID NO: 259 - DNA, 20 bp, Artificial Sequence
  Antisense oligonucleotide
tcactagtacatgagacatg

SEQ ID NO: 260 - DNA, 20 bp, Artificial Sequence
  Antisense oligonucleotide
acaaatgcacacatctactt

SEQ ID NO: 261 - DNA, 20 bp, Artificial Sequence
  Antisense oligonucleotide
ggatacatagggatatgtgc

SEQ ID NO: 262 - DNA, 20 bp, Artificial Sequence
  Antisense oligonucleotide
aatacacacagccctgatag

SEQ ID NO: 263 - DNA, 20 bp, Artificial Sequence
  Antisense oligonucleotide
tgcggacacacacactttca

SEQ ID NO: 264 - DNA, 20 bp, Artificial Sequence
  Antisense oligonucleotide
cttctatacagatatgatca

SEQ ID NO: 265 - DNA, 20 bp, Artificial Sequence
  Antisense oligonucleotide
aagaaatataatcacactct

SEQ ID NO: 266 - DNA, 20 bp, Artificial Sequence
  Antisense oligonucleotide
ttcaaatggatgtattcttc

SEQ ID NO: 267 - DNA, 20 bp, Artificial Sequence
  Antisense oligonucleotide
aaacagccatagacatccat

SEQ ID NO: 268 - DNA, 20 bp, Artificial Sequence
  Antisense oligonucleotide
aagagtagagaactcatctc

SEQ ID NO: 269 - DNA, 20 bp, Artificial Sequence
  Antisense oligonucleotide
ggagactactgtacaagcac

SEQ ID NO: 270 - DNA, 20 bp, Artificial Sequence
  Antisense oligonucleotide
accaagcataagggataagg

SEQ ID NO: 271 - DNA, 20 bp, Artificial Sequence
  Antisense oligonucleotide
tggggtctaagaacgtatcc

SEQ ID NO: 272 - DNA, 20 bp, Artificial Sequence
  Antisense oligonucleotide
tgcggtctcagagatccact

SEQ ID NO: 273 - DNA, 20 bp, Artificial Sequence
  Antisense oligonucleotide
atatgaggtttggtaccatc

SEQ ID NO: 274 - DNA, 20 bp, Artificial Sequence
  Antisense oligonucleotide
tataggaaaaaatattgcat

SEQ ID NO: 275 - DNA, 20 bp, Artificial Sequence
  Antisense oligonucleotide
ctttatcttaggtatttatg

SEQ ID NO: 276 - DNA, 20 bp, Artificial Sequence
  Antisense oligonucleotide
tgcctaattcagaagatgaa

SEQ ID NO: 277 - DNA, 20 bp, Artificial Sequence
  Antisense oligonucleotide
ttattgttaatctcttactg

SEQ ID NO: 278 - DNA, 20 bp, Artificial Sequence
  Antisense oligonucleotide
ctattcaattttattgttag

SEQ ID NO: 279 - DNA, 20 bp, Artificial Sequence
  Antisense oligonucleotide
attacaatatattattataa

SEQ ID NO: 280 - DNA, 20 bp, Artificial Sequence
  Antisense oligonucleotide
atcacattcacataactttt

SEQ ID NO: 281 - DNA, 20 bp, Artificial Sequence
  Antisense oligonucleotide
tgagagagagaaagaaagag

SEQ ID NO: 282 - DNA, 5503 bp, Homo sapiens
tttgaatacaccaattgctgtggggcggctcgaggaagagaagacaccagtgcctcagaaactgctcggtcagacggtgatagcgagccacgcattcacagggccactgctgctcacagaagcagtgaggatgatgccaggatgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctgaagaggaaaaataaaaacctcctgaggcatttcttgcccagctgggtccaacacttgttcagttaataagtattaaatgctgccacatgtcaggccttatgctaagggtgagtaattccatggtgcactagatatgcagggctgctaatctcaaggagcttccagtgcagagggaataaatgctagactaaaatacagagtcttccaggtgggcatttcaaccaactcagtcaaggaacccatgacaaagaaagtcatttcaactcttacctcatcaagttgaataaagacagagaaaacagaaagagacattgttcttttcctgagtcttttgaatggaaattgtattatgttatagccatcataaaaccattttggtagttttgactgaactgggtgttcactttttcctttttgattgaatacaatttaaattctacttgatgactgcagtcgtcaaggggctcctgatgcaagatgccccttccattttaagtctgtctccttacagaggttaaagtctagtggctaattcctaaggaaacctgattaacacatgctcacaaccatcctggtcattctcgagcatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctagaggcaggaaggaagtgggatgacctcaggaggtcaccttttcttgattccagaaacatatgggctgataaacccggggtgacctcatgaaatgagttgcagcagaagtttatttttttcagaacaagtgatgtttgatggacctctgaatctctttagggagacacagatggctgggatccctcccctgtacccttctcactgccaggagaactacgtgtgaaggtattcaaggcagggagtatacattgctgtttcctgttgggcaatgctccttgaccacattttgggaagagtggatgttatcattgagaaaacaatgtgtctggaattaatggggttcttataaagaaggttcccagaaaagaatgttcatccagcctcctcagaaacagaacattcaagaaaaggacaatcaggatgtcatcagggaaatgaaaataaaaaccacaatgagatatcaccttataccaggtagaatggctactataaaaaaatgaagtgtcatcaaggatatagagaaattggaacccttcttcactgctggagggaatggaaaatggtgtagccgttatgaaaaacagtacggaggtttctcaaaaattaaaaatagaactgctatatgatccagcaatctcacttctgtatatatacccaaaataattgaaatcagaatttcaagaaaatatttacactcccatgttcattgtggcactcttcacaatcactgtttccaaagttatggaaacaacccaaatttccattgaaaaataaatggacaaagaaaatgtgcatatacgtacaatgggatattattcagcctaaaaaaagggggaatcctgttatttatgacaacatgaataaacccggaggccattatgctatgtaaaatgagcaagtaacagaaagacaaatactgcctgatttcatttatatgaggttctaaaatagtcaaactcatagaagcagagaatagaacagtggttcctagggaaaaggaggaagggagaaatgaggaaatagggagttgtctaattggtataaaattatagtatgcaagatgaattagctctaaagatcagctgtatagcagagttcgtataatgaacaatactgtattatgcacttaacattttgttaagagggtacctctcatgttaagtgttcttaccatatacatatacacaaggaagcttttggaggtgatggatatatttattaccttgattgtggtgatggtttgacaggtatgtgactatgtctaaactcatcaaattgtatacattaaatatatgcagttttataatatcaattatgtctgaatgaagctataaaaaagaaaagacaacaaaattcagttgtcaaaactggaaatatgaccacagtcagaagtgtttgttactgagtgtttcagagtgtgtttggtttgagcaggtctagggtgattgaacatccctgggtgtgtttccatgtctcatgtactagtgaaagtagatgtgtgcatttgtgcacatatccctatgtatccctatcagggctgtgtgtatttgaaagtgtgtgtgtccgcatgatcatatctgtatagaagagagtgtgattatatttcttgaagaatacatccatttgaaatggatgtctatggctgtttgagatgagttctctactcttgtgcttgtacagtagtctccccttatcccttatgcttggtggatacgttcttagaccccaagtggatctctgagaccgcagatggtaccaaacctcatatatgcaatattttttcctatacataaatacctaagataaagttcatcttctgaattaggcacagtaagagattaacaataactaacaataaaattgaatagttataataatatattgtaataaaagttatgtgaatgtgatctctttctttctctctctcaaaa

(19) United States
(12) Patent Application Publication
(10) Pub. No.: US 2010/0111936 A1
(43) Pub. Date: May 6, 2010

(54) Modulation of Toll-Like Receptor 4 Expression by Antisense Oligonucleotides

(75) Inventors: Mallikarjuna Putta, Burlington, MA (US); Ekambar Kandimalla, Southboro, MA (US); Lakshmi Bhagat, Framingham, MA (US); Daqing Wang, Bedford, MA (US); Dong Yu, Westboro, MA (US); Sudhir Agrawal, Shrewsbury, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: IDERA PHARMACEUTICALS, INC., Cambridge, MA (US)

(21) Appl. No.: 12/612,387

(22) Filed: Nov. 4, 2009

Related U.S. Application Data
(60) Provisional application No. 61/111,148, filed on Nov. 4, 2008.

(51) Int. Cl. A61K 48/00; C07H 21/02; A61K 39/00; A61K 39/395; A61K 39/35

(52) U.S. Cl. 424/130.1; 536/23.1; 514/44 A; 424/184.1; 424/275.1

(57) ABSTRACT

Antisense oligonucleotide compounds, compositions and methods are provided for down regulating the expression of TLR4. The compositions comprise antisense oligonucleotides targeted to nucleic acids encoding TLR4. The compositions may also comprise antisense oligonucleotides targeted to nucleic acids encoding TLR4 in combination with other therapeutic and/or prophylactic compounds and/or compositions. Methods of using these compounds and compositions for down-regulating TLR4 expression and for prevention or treatment of diseases wherein modulation of TLR4 expression would be beneficial are provided.

This application claims the benefit of priority from U.S. Provisional Patent Application Ser. No. 61/111,148, filed on Nov. 4, 2008, the disclosure of which is explicitly incorporated by reference herein.

### BACKGROUND OF THE INVENTION

1. Field of the Invention

The present invention relates to Toll-Like Receptor 4 (TLR4). In particular, the invention relates to antisense oligonucleotides that specifically hybridize with nucleic acids encoding TLR4, thus modulating TLR4 expression and activity, and their use in treating or preventing diseases associated with TLR4 or wherein modulation of TLR4 expression would be beneficial.

2. Summary of the Related Art

Toll-like receptors (TLRs) are present on many cells of the immune system and have been shown to be involved in the innate immune response (Hornung, V. et al., (2002) J. Immunol. 168:4531-4537). TLRs are a key means by which mammals recognize and mount an immune response to foreign molecules and also provide a means by which the innate and adaptive immune responses are linked (Akira, S. et al. (2001) Nature Immunol. 2:675-680; Medzhitov, R. (2001) Nature Rev. Immunol. 1:135-145). In vertebrates, this family consists of at least 11 proteins called TLR1 to TLR11, which are known to recognize pathogen associated molecular patterns (PAMP) from bacteria, fungi, parasites and viruses and induce an immune response mediated by a number of transcription factors.

Some TLRs are located on the cell surface to detect and initiate a response to extracellular pathogens and other TLRs are located inside the cell to detect and initiate a response to intracellular pathogens. Table 1 provides a representation of TLRs, the known agonists therefore and the cell types known to contain the TLR (Diebold, S. S. et al. (2004) Science 303:1529-1531; Liew, F. et al. (2005) Nature 5:446-458; Hemmi H et al. (2002) Nat Immunol 3:196-200; Jurk M et al., (2002) Nat Immunol 3:499; Lee J et al. (2003) Proc. Natl. Acad. Sci. USA 100:6646-6651); (Alexopoulou, L. (2001) Nature 413:732-738).

TABLE 1

| TLR Molecule | Agonist | Cell Types Containing Receptor |
|---|---|---|
| Cell Surface TLRs: | | |
| TLR2 | bacterial lipopeptides | Monocytes/macrophages |
| | | Myeloid dendritic cells |
| | | Mast cells |
| TLR4 | gram negative bacteria | Monocytes/macrophages |
| | | Myeloid dendritic cells |
| | | Mast cells |
| | | Intestinal epithelium |
| TLR5 | motile bacteria | Monocytes/macrophages |
| | | Dendritic cells |
| | | Intestinal epithelium |
| TLR6 | gram positive bacteria | Monocytes/macrophages |
| | | Mast cells |
| | | B lymphocytes |
| Endosomal TLRs: | | |
| TLR3 | double stranded RNA viruses | Dendritic cells |
| | | B lymphocytes |
| TLR7 | single stranded RNA viruses; | Monocytes/macrophages |
| | RNA-immunoglobulin | Plasmacytoid dendritic cells |
| | complexes | B lymphocytes |
| TLR8 | single stranded RNA viruses; | Monocytes/macrophages |
| | RNA-immunoglobulin | Dendritic cells |
| | complexes | Mast cells |
| TLR9 | DNA containing unmethylated | Monocytes/macrophages |
| | “CpG” motifs; DNA- | Plasmacytoid dendritic cells |
| | immunoglobulin complexes | B lymphocytes |

The signal transduction pathway mediated by the interaction between a ligand and a TLR is shared among most members of the TLR family and involves a toll/IL-1 receptor (TIR domain), the myeloid differentiation marker 88 (MyD88), IL-1R-associated kinase (IRAK), interferon regulating factor (IRF), TNF-receptor-associated factor (TRAF), TGFβ-activated kinasel, IκB kinases, IκB, and NF-κB (see for example: Akira, S. (2003) J. Biol. Chem. 278:38105 and Geller at al. (2008) Curr. Drug Dev. Tech. 5:29-38). More specifically, for TLRs 1, 2, 4, 5, 6, 7, 8, 9 and 11, this signaling cascade begins with a PAMP ligand interacting with and activating the membrane-bound TLR, which exists as a homo-dimer in the endosomal membrane or the cell surface. Following activation, the receptor undergoes a conformational change to allow recruitment of the TIR domain containing protein MyD88, which is an adapter protein that is common to all TLR signaling pathways except TLR3. MyD88 recruits IRAK4, which phosphorylates and activates IRAK1. The activated IRAK1 binds with TRAF6, which catalyzes the addition of polyubiquitin onto TRAF6. The addition of ubiquitin activates the TAK/TAB complex, which in turn phosphorylates IRFs, resulting in NF-κB release and transport to the nucleus. NF-κB in the nucleus induces the expression of proinflammatory genes (see for example, Trinchieri and Sher (2007) Nat. Rev. Immunol. 7:179-190).

The selective localization of TLRs and the signaling generated therefrom, provides some insight into their role in the immune response. The immune response involves both an innate and an adaptive response based upon the subset of cells involved in the response. For example, the T helper (Th) cells involved in classical cell-mediated functions such as delayed-type hypersensitivity and activation of cytotoxic T lymphocytes (CTLs) are Th1 cells. This response is the body's innate response to antigen (e.g. viral infections, intracellular pathogens, and tumor cells), and results in a secretion of IFN-gamma and a concomitant activation of CTLs. TLR4 is known to localize on the cell membrane and is activated by lipids present in the cell wall of pathogens, including but not limited to lipopolysaccharides (LPS) (see for example, Aderem and Ulevitch (2000) Nature 406: 780-785). This ability of TLR4 to respond to LPS demonstrates TLR4's critical role in generating the body's innate immune response to pathogens.

As a result of their involvement in regulating an inflammatory response, TLRs have been shown to play a role in the pathogenesis of many diseases, including autoimmunity, infectious disease and inflammation (Papadimitraki et al. (2007) J. Autoimmun. 29: 310-318; Sun et al. (2007) Inflam. Allergy Drug Targets 6:223-235; Diebold (2008) Adv. Drug Deliv. Rev. 60:813-823; Cook, D. N. et al. (2004) Nature Immunol. 5:975-979; Tse and Horner (2008) Semin. Immunopathol. 30:53-62; Tobias & Curtiss (2008) Semin. Immunopathol. 30:23-27; Ropert et al. (2008) Semin. Immunopathol. 30:41-51; Lee et al. (2008) Semin. Immunopathol. 30:3-9; Gao et al. (2008) Semin. Immunopathol. 30:29-40; Vijay-Kumar et al. (2008) Semin. Immunopathol. 30:11-21). While activation of TLRs is involved in mounting an immune response, an uncontrolled or undesired stimulation of the immune system through TLRs may exacerbate certain diseases in immune compromised subjects or may cause unwanted immune stimulation. Thus, down-regulating TLR expression and/or activity may provide a useful means for disease intervention.

To date, investigative strategies aimed selectively at inhibiting TLR activity have involved small molecules (WO/2005/007672), antibodies (see for example: Duffy, K. et al. (2007) Cell Immunol. 248:103-114), catalytic RNAi technologies (e.g. small inhibitory RNAs), certain antisense molecules (Caricilli et al. (2008) J. Endocrinology 199:399), and competitive inhibition with modified or methylated oligonucleotides (see for example: Kandimalla et al. US2008/0089883; Banat and Coffman (2008) Immunol. Rev. 223:271-283). For example, chloroquine and hydroxychloroquine have been shown to block endosomal-TLR signaling by down-regulating the maturation of endosomes (Krieg, A. M. (2002) Annu Rev. Immunol. 20:709). Also, Huang et al. have shown the use of TLR4 siRNA to reverse the tumor-mediated suppression of T cell proliferation and natural killer cell activity (Huang et al. (2005) Cancer Res. 65:5009-5014), and the use of TLR9 siRNA to prevent bacterial-induced inflammation of the eye (Huang et al. (2005) Invest. Opthal. Vis. Sci. 46:4209-4216).

Additionally, several groups have used synthetic oligodeoxynucleotides having two triplet sequences, a proximal “CCT” triplet and a distal “GGG” triplet, a poly “G” (e.g. “GGGG” or “GGG”) or “GC” sequences that interact with certain intracellular proteins, resulting in the inhibition of TLR signaling and the concomitant production and release of pro-inflammatory cytokines (see for example: Lenert, P. et al. (2003) DNA Cell Biol. 22(10):621-631; Patole, P. et al. (2005) J. Am. Soc. Nephrol. 16:3273-3280), Gursel, I., et al. (J. Immunol., 171: 1393-1400 (2003), Shirota, H., et al., J. Immunol., 173: 5002-5007 (2004), Chen, Y., et al., Gene Ther. 8: 1024-1032 (2001); Stunz, L. L., Eur. J. Immunol. (2002) 32: 1212-1222; Kandimalla et al. WO2007/7047396). However, oligonucleotides containing guanosine strings have been shown to form tetraplex structures, act as aptamers and inhibit thrombin activity (Bock L C et al., Nature, 355:564-6, 1992; Padmanabhan, K et al., J Biol. Chem., 268(24):17651-4, 1993). Thus, the utility of these inhibitory oligodeoxynucleotide molecules may not be achievable in patients.

A potential approach to “inhibiting, suppressing, or down-regulating” expression of TLRs is antisense technology. The history of developing antisense technology indicates that while designing and testing of antisense oligonucleotides that hybridize to target RNA is a relatively straight forward exercise, only a few antisense oligonucleotides work as intended and optimization of antisense oligonucleotides that have true potential as clinical candidates is not predictable. One skilled in the art would recognize that when optimizing antisense oligonucleotides, conceiving the correct oligonucleotide sequence and length, and utilizing the appropriate nucleic acid and oligonucleotide chemistries are not readily apparent. However, formulating these components is crucial to the utility of any antisense oligonucleotide (Stein and Cheng, 1993, Science 261: 1004-1012). One skilled in the art would further recognize that without conceiving the correct sequence, the correct length, and utilizing the appropriate nucleic acid and oligonucleotide chemistries, the antisense oligonucleotide can have off-target effects and can cause, among other things, the molecule to be unstable, inactive, non-specific, and toxic. As a result of the unpredictable nature of antisense oligonucleotides, to date only one antisense oligonucleotide has received approval for use in humans, and no antisense oligonucleotides are currently being marketed for human use.

Accordingly, there exists a need in the field for optimized antisense oligonucleotides that most efficiently down-regulate or inhibit gene expression. In particular, there exists a need in the field for antisense oligonucleotides that down-regulate TLR4 expression and that are stable, active, target specific, non-toxic, and do not activate an innate immune response. A molecule with such characteristics would overcome the problems that have previously prevented antisense oligonucleotides from being developed.

### BRIEF SUMMARY OF THE INVENTION

The present invention is directed to, among other things, optimized synthetic antisense oligonucleotides that are targeted to a nucleic acid encoding TLR4 and that efficiently inhibit the expression of TLR4 through inhibition of mRNA translation and/or through an RNase H mediated mechanism.

In a first aspect, optimized antisense oligonucleotides according to the invention include those having SEQ ID NOs: 7, 8, 17, 24, 30, 49, 86, 100, 102, 115, 121, 126, 136, 146, 184 or 256.

In another aspect, the invention provides a composition comprising at least one optimized antisense oligonucleotide according to the invention and a physiologically acceptable carrier, diluent or excipient.

In another aspect, the invention provides a method of inhibiting TLR4 expression. In this method, an oligonucleotide or multiple oligonucleotides of the invention are specifically contacted or hybridized with TLR4 mRNA either in vitro or in a cell.

In another aspect, the invention provides methods for inhibiting the expression of TLR4 in a mammal, particularly a human, such methods comprising administering to the mammal a compound or composition according to the invention.

In another aspect, the invention provides a method for inhibiting a TLR4-mediated immune response in a mammal, the method comprising administering to the mammal a TLR4 antisense oligonucleotide according to the invention in a pharmaceutically effective amount.

In another aspect, the invention provides a method for therapeutically treating a mammal having a disease mediated by TLR4, such method comprising administering to the mammal, particularly a human, a TLR4 antisense oligonucleotide of the invention, or a composition thereof, in a pharmaceutically effective amount.

In another aspect, the invention provides methods for preventing a disease or disorder in a mammal, particularly a human, at risk of contracting or developing a disease or disorder mediated by TLR4. Such methods comprise administering to the mammal an antisense oligonucleotide according to the invention, or a composition thereof, in a prophylactically effective amount.

In another aspect, the invention provides a method for inhibiting TLR4 expression and activity in a mammal, comprising administering to the mammal an antisense oligonucleotide complementary to TLR4 mRNA and an antagonist of TLR4 protein, a kinase inhibitor or an inhibitor of signal transduction and transcription (STAT) protein.

The subject oligonucleotides and methods disclosed herein are also useful for examining the function of the TLR4 gene in a cell or in a control mammal or in a mammal afflicted with a disease or disorder associated with TLR4 or immune stimulation through TLR4. The cell or mammal is administered the oligonucleotide, and the expression of TLR4 mRNA or protein is examined.

### BRIEF DESCRIPTION OF THE DRAWINGS

FIG. 1 is a synthetic scheme for the linear synthesis of antisense oligonucleotides of the invention. DMTr=4,4′-dimethoxytrityl; CE=cyanoethyl.

FIG. 2 demonstrates that exemplary human TLR4 antisense oligonucleotides according to the invention are not immunostimulatory (Antisense Alone). FIG. 2 also demonstrates the ability of exemplary oligonucleotides according to the invention to inhibit TLR4 expression and activation in HEK293 cells that were cultured and treated according to Example 2 (Agonist plus Antisense).

FIG. 3 shows the nucleotide sequence of humanTLR4 mRNA [SEQ ID NO: 282] (Genbank Accession No. NM 138554).

### DETAILED DESCRIPTION

The invention relates to optimized TLR4 antisense oligonucleotides, compositions comprising such oligonucleotides and methods of their use for inhibiting or suppressing a TLR4-mediated immune response. More specifically, the antisense oligonucleotides according to the invention are stable, active, target specific, non-toxic, and do not activate an innate immune response. Pharmaceutical and other compositions comprising the compounds according to the invention are also provided. Further provided are methods of down-regulating the expression of TLR4 in cells or tissues comprising contacting said cells or tissues with one or more of the antisense compounds or compositions of the invention alone or in combination with other prophylactic or therapeutic compositions.

Specifically, the invention provides antisense oligonucleotides designed to be complementary to a genomic region or an RNA molecule transcribed therefrom. These TLR4 antisense oligonucleotides are stable, target specific, and have unique sequences that result in the molecule being maximally effective at inhibiting or suppressing TLR4-mediated signaling in response to endogenous and/or exogenous TLR4 ligands or TLR4 agonists.

The TLR4 antisense oligonucleotides according to the invention inhibit immune responses induced by natural or artificial TLR4 agonists in various cell types and in various in vitro and in vivo experimental models. As such, the antisense compositions according to the invention are useful as tools to study the immune system, as well as to compare the immune systems of various mammals, such as humans and mice.

Further provided are methods of treating a mammal, particularly a human, having, suspected of having, or being prone to develop a disease or condition associated with TLR4 activation by administering a therapeutically or prophylactically effective amount of one or more of the antisense compounds or compositions of the invention. Since TLR4 has been identified as an important initiator of proinflammatory responses, whose activity has been correlated to several diseases (see for example: Gribar et al. (2008) J. Leukoc. Biol. 83:493-498; Fukata and Abreu (2007) Biochem. Soc. Trans. 35: 1473-1478; Gao et al. (2007) Curr. Opin. Allergy Clin. Immunol. 7:459-467), the optimized antisense oligonucleotides and compositions according to the invention can be used for immunotherapy applications such as, but not limited to, treatment of cancer, autoimmune disorders, asthma, respiratory allergies, food allergies, skin allergies, systemic lupus erythematosus (SLE), arthritis, pleurisy, chronic infections, inflammatory diseases, inflammatory bowel syndrome, sepsis, malaria, and bacteria, parasitic, and viral infections in adult and pediatric human and veterinary applications. In addition, TLR4 antisense oligonucleotides of the invention are useful in the prevention and/or treatment of various diseases, either alone, in combination with or co-administered with other drugs or prophylactic or therapeutic compositions, for example, DNA vaccines, antigens, antibodies, and allergens; and in combination with chemotherapeutic agents (both traditional chemotherapy and modern targeted therapies) and/or TLR4 antagonists for prevention and treatment of diseases. TLR4 antisense oligonucleotides of the invention are useful in combination with compounds or drugs that have unwanted TLR4-mediated immune stimulatory properties.

The objects of the present invention, the various features thereof, as well as the invention itself may be more fully understood from the following description, when read together with the accompanying drawings in which the following terms have the ascribed meaning:

The term “2′-O-substituted” means substitution of the 2′ position of the pentose moiety with an —O— lower alkyl group containing 1-6 saturated or unsaturated carbon atoms (for example, but not limited to, 2′-O-methyl), or with an —O-aryl or allyl group having 2-6 carbon atoms, wherein such alkyl, aryl or allyl group may be unsubstituted or may be substituted, (for example, with 2′-O-ethoxy-methyl, halo, hydroxy, trifluoromethyl, cyano, nitro, acyl, acyloxy, alkoxy, carboxyl, carbalkoxyl, or amino groups); or with a hydroxy, an amino or a halo group, but not with a 2′-H group. In some embodiments the oligonucleotides of the invention include four or five 2′-O-alkyl ribonucleotides at their 5′ terminus, and/or four or five 2′-O-alkyl ribonucleotides at their 3′ terminus. In exemplary embodiments, the nucleotides of the synthetic oligonucleotides are linked by at least one phosphorothioate internucleotide linkage. The phosphorothioate linkages may be mixed Rp and Sp enantiomers, or they may be stereoregular or substantially stereoregular in either Rp or Sp form (see Iyer et al. (1995) Tetrahedron Asymmetry 6:1051-1054).

The term “3′”, when used directionally, generally refers to a region or position in a polynucleotide or oligonucleotide 3′ (toward the 3′ end of the nucleotide) from another region or position in the same polynucleotide or oligonucleotide.

The term “5′”, when used directionally, generally refers to a region or position in a polynucleotide or oligonucleotide 5′ (toward the 5′ end of the nucleotide) from another region or position in the same polynucleotide or oligonucleotide.

The term “about” generally means that the exact number is not critical. Thus, oligonucleotides having one or two fewer nucleoside residues, or from one to several additional nucleoside residues are contemplated as equivalents of each of the embodiments described above.

The term “agonist” generally refers to a substance that binds to a receptor of a cell and induces a response. An agonist often mimics the action of a naturally occurring substance such as a ligand.

The term “antagonist” generally refers to a substance that attenuates the effects of an agonist.

The term “airway inflammation” generally includes, without limitation, inflammation in the respiratory tract caused by allergens, including asthma.

The term “allergen” generally refers to an antigen or antigenic portion of a molecule, usually a protein, which elicits an allergic response upon exposure to a subject. Typically the subject is allergic to the allergen as indicated, for instance, by the wheal and flare test or any method known in the art. A molecule is said to be an allergen even if only a small subset of subjects exhibit an allergic (e.g., IgE) immune response upon exposure to the molecule.

The term “allergy” generally includes, without limitation, food allergies, respiratory allergies and skin allergies.

The term “antigen” generally refers to a substance that is recognized and selectively bound by an antibody or by a T cell antigen receptor. Antigens may include but are not limited to peptides, proteins, nucleosides, nucleotides and combinations thereof. Antigens may be natural or synthetic and generally induce an immune response that is specific for that antigen.

The term “autoimmune disorder” generally refers to disorders in which “self” antigen undergo attack by the immune system. Such term includes, without limitation, lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis autoimmune asthma, septic shock and psoriasis.

The term “cancer” generally refers to, without limitation, any malignant growth or tumor caused by abnormal or uncontrolled cell proliferation and/or division. Cancers may occur in humans and/or mammals and may arise in any and all tissues. Treating a patient having cancer may include administration of a compound, pharmaceutical formulation or vaccine according to the invention such that the abnormal or uncontrolled cell proliferation and/or division, or metastasis is affected.

The term “carrier” generally encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, for example, Remington's Pharmaceutical Sciences, 18th Edition, ed. A. Gennaro, Mack Publishing Co., Easton, Pa., 1990.

The terms “co-administration” or “co-administered” generally refer to the administration of at least two different substances sufficiently close in time to modulate an immune response. Co-administration refers to simultaneous administration, as well as temporally spaced order of up to several days apart, of at least two different substances in any order, either in a single dose or separate doses.

The term “in combination with” generally means administering a compound according to the invention and another agent useful for treating the disease or condition that does not abolish TLR4 antisense activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The terms “individual” or “subject” or “vertebrate” or “patient” generally refer to a mammal, such as a human.

The terms “inhibit” or “down regulate” or “suppress”, when used in reference to expression, generally refer to a decrease in a response or qualitative difference in a response, which could otherwise arise from eliciting and/or stimulation of a response.

The term “kinase inhibitor” generally refers to molecules that antagonize or inhibit phosphorylation-dependent cell signaling and/or growth pathways in a cell. Kinase inhibitors may be naturally occurring or synthetic and include small molecules that have the potential to be administered as oral therapeutics. Kinase inhibitors have the ability to rapidly and specifically inhibit the activation of the target kinase molecules. Protein kinases are attractive drug targets, in part because they regulate a wide variety of signaling and growth pathways and include many different proteins. As such, they have great potential in the treatment of diseases involving kinase signaling, including cancer, cardiovascular disease, inflammatory disorders, diabetes, macular degeneration and neurological disorders. Examples of kinase inhibitors include, but are not limited to, sorafenib (Nexavar®), Sutent®, dasatinib, Dasatinib™, Zactima™, Tykerb™ and STI571.

The term “linear synthesis” generally refers to a synthesis that starts at one end of an oligonucleotide and progresses linearly to the other end. Linear synthesis permits incorporation of either identical or non-identical (in terms of length, base composition and/or chemical modifications incorporated) monomeric units into an oligonucleotide.

The term “mammal” is expressly intended to include warm blooded, vertebrate animals, including, without limitation, humans, non-human primates, rats, mice, cats, dogs, horses, cattle, cows, pigs, sheep and rabbits.

The term “nucleoside” generally refers to compounds consisting of a sugar, usually ribose or deoxyribose, and a purine or pyrimidine base.

The term “nucleotide” generally refers to a nucleoside comprising a phosphorous-containing group attached to the sugar.

The term “modified nucleoside” generally is a nucleoside that includes a modified heterocyclic base, a modified sugar moiety, or any combination thereof. In some embodiments, the modified nucleoside is a non-natural pyrimidine or purine nucleoside, as herein described. For purposes of the invention, a modified nucleoside, a pyrimidine or purine analog or non-naturally occurring pyrimidine or purine can be used interchangeably and refers to a nucleoside that includes a non-naturally occurring base and/or non-naturally occurring sugar moiety. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “modified oligonucleotide” as used herein describes an oligonucleotide in which at least two of its nucleotides are covalently linked via a synthetic linkage, i.e., a linkage other than a phosphodiester linkage between the 5′ end of one nucleotide and the 3′ end of another nucleotide in which the 5′ nucleotide phosphate has been replaced with any number of chemical groups. The term “modified oligonucleotide” also encompasses oligonucleotides having at least one nucleotide with a modified base and/or sugar, such as a 2′-O-substituted, a 5-methylcytosine and a 3′-O-substituted ribonucleotide.

The term “nucleic acid” encompasses a genomic region or an RNA molecule transcribed therefrom. In some embodiments, the nucleic acid is mRNA.

The term “nucleotidic linkage” generally refers to a chemical linkage to join two nucleosides through their sugars (e.g. 3′-3′, 2′-3′,2′-5′, 3′-5′, 5′-5′) consisting of a phosphorous atom and a charged, or neutral group (e.g., phosphodiester, phosphorothioate, phosphorodithioate or methylphosphonate) between adjacent nucleosides.

The term “oligonucleotide” refers to a polynucleoside formed from a plurality of linked nucleoside units. The nucleoside units may be part of viruses, bacteria, cell debris or oligonucleotide-based compositions (for example, siRNA and microRNA). Such oligonucleotides can also be obtained from existing nucleic acid sources, including genomic or cDNA, but are preferably produced by synthetic methods. In certain embodiments each nucleoside unit includes a heterocyclic base and a pentofuranosyl, trehalose, arabinose, 2′-deoxy-2′-substituted nucleoside, 2′-deoxy-2′-substituted arabinose, 2′-O-substitutedarabinose or hexose sugar group. The nucleoside residues can be coupled to each other by any of the numerous known internucleoside linkages. Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide-based compound” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain exemplary embodiments, these internucleoside linkages may be phosphodiester, phosphorothioate or phosphorodithioate linkages, or combinations thereof.

The term “complementary to a genomic region or an RNA molecule transcribed therefrom” is intended to mean an oligonucleotide that binds to the nucleic acid sequence under physiological conditions, for example, by Watson-Crick base pairing (interaction between oligonucleotide and single-stranded nucleic acid) or by Hoogsteen base pairing (interaction between oligonucleotide and double-stranded nucleic acid) or by any other means, including in the case of an oligonucleotide, binding to RNA and causing pseudoknot formation. Binding by Watson-Crick or Hoogsteen base pairing under physiological conditions is measured as a practical matter by observing interference with the function of the nucleic acid sequence.

The term “peptide” generally refers to polypeptides that are of sufficient length and composition to affect a biological response, for example, antibody production or cytokine activity whether or not the peptide is a hapten. The term “peptide” may include modified amino acids (whether or not naturally or non-naturally occurring), where such modifications include, but are not limited to, phosphorylation, glycosylation, pegylation, lipidization and methylation.

The term “pharmaceutically acceptable” means a non-toxic material that does not interfere with the effectiveness of a compound according to the invention or the biological activity of a compound according to the invention.

The term “physiologically acceptable” refers to a non-toxic material that is compatible with a biological system such as a cell, cell culture, tissue, or organism. Preferably, the biological system is a living organism, such as a mammal, particularly a human.

The term “prophylactically effective amount” generally refers to an amount sufficient to prevent or reduce the development of an undesired biological effect.

The terms “therapeutically effective amount” or “pharmaceutically effective amount” generally refer to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful patient benefit, for example, but not limited to, healing of chronic conditions characterized by immune stimulation. Thus, a “pharmaceutically effective amount” will depend upon the context in which it is being administered. A pharmaceutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “treatment” generally refers to an approach intended to obtain a beneficial or desired result, which may include alleviation of symptoms, or delaying or ameliorating a disease progression.

The invention provides antisense oligonucleotides that are complementary to a nucleic acid that is specific for human TLR4 (SEQ ID NO: 282). The antisense oligonucleotides according to the invention are optimized with respect to (i) the targeted region of the TLR4 mRNA coding sequence, the 5′ untranslated region or the 3′ untranslated region, (ii) their chemical modification(s), or (iii) both. In some embodiments, the compounds are complementary to a region within nucleotides 142 through 2661 of the coding region, or nucleotides 1-141 of the 5′ untranslated region, or 2662-5503 of the 3′ untranslated region of TLR4 mRNA (SEQ ID NO: 282).

Antisense oligonucleotides according to the invention are useful in treating and/or preventing diseases wherein inhibiting a TLR4-mediated immune response would be beneficial. TLR4-targeted antisense oligonucleotides according to the invention that are useful include, but are not limited to, antisense oligonucleotides comprising naturally occurring nucleotides, modified nucleotides, modified oligonucleotides and/or backbone modified oligonucleotides. However, antisense oligonucleotides that inhibit the translation of mRNA encoded proteins may produce undesired biological effects, including but not limited to insufficiently active antisense oligonucleotides, inadequate bioavailability, suboptimal pharmacokinetics or pharmacodynamics, and immune stimulation. Thus, the optimal design of an antisense oligonucleotide according to the invention requires many considerations beyond simple design of a complementary sequence. Thus, preparation of TLR4-targeted antisense oligonucleotides according to the invention is intended to incorporate changes necessary to limit secondary structure interference with antisense activity, enhance the oligonucleotide's target specificity, minimize interaction with binding or competing factors (for example, proteins), optimize cellular uptake, stability, bioavailability, pharmacokinetics and pharmacodynamics, and/or inhibit, prevent or suppress immune cell activation.

It has been determined that the human TLR4 genes is expressed as 4 kb, 5 kb and 7 kb transcripts that are expressed in a tissue specific manner (Medzhitov et al. (1997) Nature 388:394-397; Rock et al. (1998) Proc. Nat. Acad. Sci. 95:588-593) that is most abundant in endothelial cells, B cells, and myeloid cells. The transcripts contain a 2.5 kb coding region, which encodes an 841 amino acid protein in humans. The oligonucleotides of the invention were designed to specifically hybridize with optimally available portions of the TLR4 nucleic acid sequence that most effectively act as a target for inhibiting TLR4 expression. These targeted regions of the TLR4 gene include portions of the known exons or 5′ untranslated region. In addition, intron-exon boundaries, 3′ untranslated regions and introns are potentially useful targets for antisense inhibition of TLR4 expression. The nucleotide sequences of some representative, non-limiting oligonucleotides specific for human TLR4 have SEQ ID NOS: 1-281. The nucleotide sequences of optimized oligonucleotides according to the invention include those having SEQ ID NOS: 7, 8, 17, 24, 30, 49, 86, 100, 102, 115, 121, 126, 136, 146, 184 or 256.

The oligonucleotides of the invention are at least 14 nucleotides in length, but are preferably 15 to 60 nucleotides long, preferably 20 to 50 nucleotides in length. In some embodiments, these oligonucleotides contain from about 14 to 28 nucleotides or from about 16 to 25 nucleotides or from about 18 to 22 nucleotides or 20 nucleotides. These oligonucleotides can be prepared by the art recognized methods such as phosphoramidate or H-phosphonate chemistry which can be carried out manually or by an automated synthesizer. The synthetic TLR4 antisense oligonucleotides of the invention may also be modified in a number of ways without compromising their ability to hybridize to TLR4 mRNA. Such modifications may include at least one internucleotide linkage of the oligonucleotide being an alkylphosphonate, phosphorothioate, phosphorodithioate, methyl phosphonate, phosphate ester, alkylphosphonothioate, phosphoramidate, carbamate, carbonate, phosphate triester, acetamidate or carboxymethyl ester or a combination of these and other internucleotide linkages between the 5′ end of one nucleotide and the 3′ end of another nucleotide in which the 5′ nucleotide phosphodiester linkage has been replaced with any number of chemical groups.

For example, U.S. Pat. No. 5,149,797 describes traditional chimeric oligonucleotides having a phosphorothioate core region interposed between methylphosphonate or phosphoramidate flanking regions. U.S. Pat. No. 5,652,356 discloses “inverted” chimeric oligonucleotides comprising one or more nonionic oligonucleotide region (e.g. alkylphosphonate and/or phosphoramidate and/or phosphotriester internucleoside linkage) flanked by one or more region of oligonucleotide phosphorothioate. Various oligonucleotides with modified internucleotide linkages can be prepared according to standard methods. Phosphorothioate linkages may be mixed Rp and Sp enantiomers, or they may be made stereoregular or substantially stereoregular in either Rp or Sp form according to standard procedures.

Oligonucleotides which are self-stabilized are also considered to be modified oligonucleotides useful in the methods of the invention (Tang et al. (1993) Nucleic Acids Res. 20:2729-2735). These oligonucleotides comprise two regions: a target hybridizing region; and a self-complementary region having an oligonucleotide sequence complementary to a nucleic acid sequence that is within the self-stabilized oligonucleotide.

Other modifications include those which are internal or at the end(s) of the oligonucleotide molecule and include additions to the molecule of the internucleoside phosphate linkages, such as cholesterol, cholesteryl, or diamine compounds with varying numbers of carbon residues between the amino groups and terminal ribose, deoxyribose and phosphate modifications which cleave, or crosslink to the opposite chains or to associated enzymes or other proteins which bind to the genome. Examples of such modified oligonucleotides include oligonucleotides with a modified base and/or sugar such as arabinose instead of ribose, or a 3′, 5′-substituted oligonucleotide having a sugar which, at both its 3′ and 5′ positions, is attached to a chemical group other than a hydroxyl group (at its 3′ position) and other than a phosphate group (at its 5′ position).

Other examples of modifications to sugars include modifications to the 2′ position of the ribose moiety which include but are not limited to 2′-O-substituted with an —O-alkyl group containing 1-6 saturated or unsaturated carbon atoms, or with an —O-aryl, or O-allyl group having 2-6 carbon atoms wherein such —O-alkyl, —O-aryl or O-allyl group may be unsubstituted or may be substituted, for example with halo, hydroxy, trifluoromethyl cyano, nitro acyl acyloxy, alkoxy, carboxy, carbalkoxyl or amino groups. None of these substitutions are intended to exclude the native 2′-hydroxyl group in the case of ribose or 2′1-H— in the case of deoxyribose.

The oligonucleotides according to the invention can comprise one or more ribonucleotides. For example, U.S. Pat. No. 5,652,355 discloses traditional hybrid oligonucleotides having regions of 2′-O-substituted ribonucleotides flanking a DNA core region. U.S. Pat. No. 5,652,356 discloses an “inverted” hybrid oligonucleotide which includes an oligonucleotide comprising a 2′-O-substituted (or 2′ OH, unsubstituted) RNA region which is in between two oligodeoxyribonucleotide regions, a structure that “inverted relative to the “traditional” hybrid oligonucleotides. Non-limiting examples of particularly useful oligonucleotides of the invention have 2′-O-alkylated ribonucleotides at their 3′, 5′, or 3′ and 5′ termini, with at least four or five contiguous nucleotides being so modified. Non-limiting examples of 2′-O-alkylated groups include 2′-O-methyl, 2′-O-ethyl, 2′-O-propyl, 2′-O-butyls and 2′-O-methoxy-ethyl.

Other modified oligonucleotides are capped with a nuclease resistance-conferring bulky substituent at their 3′ and/or 5′ end(s), or have a substitution in one non-bridging oxygen per nucleotide. Such modifications can be at some or all of the internucleoside linkages, as well as at either or both ends of the oligonucleotide and/or in the interior of the molecule.

The oligonucleotides of the invention can be administered in combination with one or more antisense oligonucleotides or other nucleic acid containing compounds that are not targeted to the same region as the antisense molecule of the invention. Such other nucleic acid containing compounds include, but are not limited to, ribozymes, RNAi molecules, siRNA, miRNA, and aptamers. In addition, the oligonucleotides of the invention can be administered in combination with one or more compounds or compositions that would activate a TLR4-mediated immune response but for the presence of the TLR4 antisense oligonucleotide according to the invention. In addition, the oligonucleotides of the invention can be administered in combination with one or more vaccines, antigens, antibodies, cytotoxic agents, allergens, antibiotics, TLR antagonists, siRNA, miRNA, antisense oligonucleotides, aptamers, peptides, proteins, gene therapy vectors, DNA vaccines, adjuvants, kinase inhibitors, inhibitors of STAT protein, or co-stimulatory molecules or combinations thereof.

A non-limiting list of TLR4 antisense oligonucleotides are shown in SEQ ID NO. 1 through SEQ ID NO. 281 and Table 2 below. Optimized antisense oligonucleotides according to the invention include those having SEQ ID NOS: 7, 8, 17, 24, 30, 49, 86, 100, 102, 115, 121, 126, 136, 146, 184 or 256. In Table 2, the oligonucleotide-based TLR4 antisense compounds have all phosphorothioate (PS) linkages. Those skilled in the art will recognize, however, that phosphodiester (PO) linkages, or a mixture of PS and PO linkages can be used.

TABLE 2

| SEQ ID NO./ | Position | Antisense Sequence |
|---|---|---|
| AS NO. | of Binding | Orientation is 5′-3′ |
| 1 | 1 | CAGCAATTGGTGTATTCAAA |
| 2 | 21 | CTCTTCCTCGAGCCGCCCCA |
| 3 | 41 | TTCTGAGGCACTGGTGTCTT |
| 4 | 61 | TCACCGTCTGACCGAGCAGT |
| 5 | 81 | TGTGAATGCGTGGCTCGCTA |
| 6 | 101 | TCTGTGAGCAGCAGTGGCCC |
| 7 | 119 | GGCATCATCCTCACTGCUUC |
| 8 | 134 | GGCAGACATCATCCTGGCAU |
| 9 | 141 | GGCGCGAGGCAGACATCATC |
| 10 | 161 | TGGGATCAGAGTCCCAGCCA |
| 11 | 181 | CAGGAGAGGAAGGCCATGGC |
| 12 | 201 | CCCAGCTTTCTGGTCTCACG |
| 13 | 221 | AACCACCTCCACGCAGGGCT |
| 14 | 241 | CATTGATAAGTAATATTAGG |
| 15 | 261 | TGTAGAAATTCAGCTCCATG |
| 16 | 281 | GGGGAGGTTGTCGGGGATTT |
| 17 | 307 | CUCAGGTCCAGGTTCTUGGU |
| 18 | 321 | TCAGGGGATTAAAGCTCAGG |
| 19 | 341 | GCTATAGCTGCCTAAATGCC |
| 20 | 361 | AGTTCTGGGAAACTGAAGAA |
| 21 | 381 | TGGATAAATCCAGCACCTGC |
| 22 | 401 | AATTGTCTGGATTTCACACC |
| 23 | 421 | CTCTGATATGCCCCATCTTC |
| 24 | 429 | GGCUTAGGCTCTGATAUGCC |
| 25 | 441 | AGGTAGAGAGGTGGCTTAGG |
| 26 | 461 | GGGGTTTCCTGTCAATATTA |
| 27 | 481 | CCCAGGGCTAAACTCTGGAT |
| 28 | 501 | TTGATAGTCCAGAAAAGGCT |
| 29 | 521 | AGCCACCAGCTTCTGTAAAC |
| 30 | 528 | UCUCCACAGCCACCAGCUUC |
| 31 | 541 | GATGCTAGATTTGTCTCCAC |
| 32 | 561 | CAATGGGGAAGTTCTCTAGA |
| 33 | 581 | TTTCAAAGTTTTGAGATGTC |
| 34 | 601 | TTGTGAGCCACATTAAGTTC |
| 35 | 621 | ATTTGAAAGATTGGATAAGA |
| 36 | 641 | ATTAGAAAAATACTCAGGTA |
| 37 | 661 | AAGTGCTCTAGATTGGTCAG |
| 38 | 681 | TCTTGTTGCTGGAAAGGTCC |
| 39 | 701 | TGTGCAATAAATACTTTGAA |
| 40 | 721 | TGATGTAGAACCCGCAAGTC |
| 41 | 741 | AGAGATTGAGTAGGGGCATT |
| 42 | 761 | GTTCAGGGACAGGTCTAAAG |
| 43 | 781 | GGTTGGATAAAGTTCATAGG |
| 44 | 801 | TAATTTCTTTAAATGCACCT |
| 45 | 821 | TAAAGTCAGCTTATGAAGCC |
| 46 | 841 | AAACTATCAAAATTATTTCT |
| 47 | 861 | TACAAGTTTTCATTACATTT |
| 48 | 881 | TAAACCAGCCAGACCTTGAA |
| 49 | 891 | GAUGGACTTCTAAACCAGCC |
| 50 | 901 | AGAACCAAACGATGGACTTC |
| 51 | 921 | CTTCATTTCTAAATTCTCCC |
| 52 | 941 | GTCAAACTTTTCCAAGTTTC |
| 53 | 961 | AGGCCCTCTAGAGCAGATTT |
| 54 | 981 | CTTCAATGGTCAAATTGCAC |
| 55 | 1001 | TAAGTATGCTAATCGGAATT |
| 56 | 1021 | ATATCATCGAGGTAGTAGTC |
| 57 | 1041 | AACAATTAAATAAGTCAATA |
| 58 | 1061 | AAATGAAGAAACATTTGTCA |
| 59 | 1081 | ATAGTCACACTCACCAGGGA |
| 60 | 1101 | AAAAGTCTTTTACCCTTTCA |
| 61 | 1121 | TTGCCATCCGAAATTATAAG |
| 62 | 1141 | CAGTTAACTAATTCTAAATG |
| 63 | 1161 | TGGGAAACTGTCCAAATTTA |
| 64 | 1181 | GAGAGATTTGAGTTTCAATG |
| 65 | 1201 | GAAGTGAAAGTAAGCCTTTT |
| 66 | 1221 | AAGCATTCCCACCTTTGTTG |
| 67 | 1241 | TGGTAGATCAACTTCTGAAA |
| 68 | 1261 | AGATCTAGAAACTCAAGGCT |
| 69 | 1281 | AACTCAAGCCATTTCTACTG |
| 70 | 1301 | TTGAGAACAGCAACCTTTGA |
| 71 | 1321 | CTGGTTGTCCCAAAATCACT |
| 72 | 1341 | TCAGATCTAAATACTTTAGG |
| 73 | 1361 | GGTAATAACACCATTGAAGC |
| 74 | 1381 | CCCAAGAAGTTTGAACTCAT |
| 75 | 1401 | GATGTTCTAGTTGTTCTAAG |
| 76 | 1421 | ATTGGAATGCTGGAAATCCA |
| 77 | 1441 | AACTCACTCATTTGTTTCAA |
| 78 | 1461 | TGAGTGATAGGAATACTGAA |
| 79 | 1481 | GTCAAGGTAAATGAGGTTTC |
| 80 | 1501 | CTGGTGTGAGTATGAGAAAT |
| 81 | 1521 | AGATGCCATTGAAAGCAACT |
| 82 | 1541 | GAGACTGGACAAGCCATTGA |
| 83 | 1561 | CCAGCCATTTTCAAGACTTC |
| 84 | 1581 | AGTTTTCCTGGAAAGAATTG |
| 85 | 1601 | TGTGAAGATATCTGGAAGGA |
| 86 | 1626 | CCAGGAAGGTCAAGTTUCUC |
| 87 | 1641 | GACACTGAGAGAGGTCCAGG |
| 88 | 1661 | TGGAGACAACTGCTCCAGTT |
| 89 | 1681 | GAGAGTGAGTTAAATGCTGT |
| 90 | 1701 | TATTTAGTACCTGAAGACTG |
| 91 | 1721 | AAAGAAGTTGTTGTGGCTCA |
| 92 | 1741 | TAAGGAAACGTATCCAATGA |
| 93 | 1761 | GGAGGGAGTTCAGACACTTA |
| 94 | 1781 | GAGACTGTAATCAAGAACCT |
| 95 | 1801 | TTGGAAGTCATTATGTGATT |
| 96 | 1821 | AATGCTGTAGTTCCTGTTTT |
| 97 | 1841 | GAAAGCTAGACTACTTGGAA |
| 98 | 1861 | TCATTCTGAGTAAGATTTAA |
| 99 | 1881 | GTTCACAAGTACAAGCAAAG |
| 100 | 1901 | CCAUTGCAGGAAACTCUGGU |
| 101 | 1914 | TCTGGTCCTTGATCCATTGC |
| 102 | 1926 | CCAAGAGCTGCCTCTGGUCC |
| 103 | 1941 | TTCGTTCAACTTCCACCAAG |
| 104 | 1962 | CTGAAGGTGTTGCACATTCC |
| 105 | 1981 | ACAGGCATGCCCTGCTTATC |
| 106 | 2001 | AGGTGATATTCAAACTCAGC |
| 107 | 2021 | GATGGTCTTATTCATCTGAC |
| 108 | 2041 | CTGAGGACCGACACACCAAT |
| 109 | 2061 | CAACAGATACTACAAGCACA |
| 110 | 2081 | CTTATAGACCAGAACTGCTA |
| 111 | 2101 | AGCATCAGGTGAAAATAGAA |
| 112 | 2121 | ACTTTATGCAGCCAGCAAGA |
| 113 | 2141 | GATGTTTTCACCTCTACCAT |
| 114 | 2161 | TAGATAACAAAGGCATCATA |
| 115 | 2182 | CAGUCCTCATCCTGGCUUGA |
| 116 | 2201 | TACTAGCTCATTCCTTACCC |
| 117 | 2221 | ACCCCTTCTTCTAAATTCTT |
| 118 | 2241 | GGCAGAGCTGAAATGGAGGC |
| 119 | 2261 | AATAAAGTCTCTGTAGTGAA |
| 120 | 2281 | GCAGCAATGGCCACACCGGG |
| 121 | 2299 | CCUUCATGGATGATGTUGGC |
| 122 | 2321 | CACCTTTCGGCTTTTATGGA |
| 123 | 2341 | TGCTGGGACACCACAACAAT |
| 124 | 2361 | ACCAGCGGCTCTGGATGAAG |
| 125 | 2381 | AATCTCATATTCAAAGATAC |
| 126 | 2406 | UGCUCAGAAACTGCCAGGUC |
| 127 | 2421 | TGATACCAGCACGACTGCTC |
| 128 | 2441 | CTTCTGCAGGACAATGAAGA |
| 129 | 2461 | CTGAGCAGGGTCTTCTCCAC |
| 130 | 2481 | GGTACAGCTCCACCTGCTGC |
| 131 | 2501 | AGTGTTCCTGCTGAGAAGGC |
| 132 | 2521 | CTGTCCTCCCACTCCAGGTA |
| 133 | 2541 | AGATGTGCCGCCCCAGGACA |
| 134 | 2561 | TTTTCTGAGTCGTCTCCAGA |
| 135 | 2581 | GATTTACCATCCAGCAGGGC |
| 136 | 2603 | CACUGTTCCTTCTGGAUUCC |
| 137 | 2621 | CCAATTGCATCCTGTACCCA |
| 138 | 2641 | CAGATAGATGTTGCTTCCTG |
| 139 | 2661 | AGGTTTTTATTTTTCCTCTT |
| 140 | 2681 | TGGGCAAGAAATGCCTCAGG |
| 141 | 2701 | GAACAAGTGTTGGACCCAGC |
| 142 | 2721 | GCATTTAATACTTATTAACT |
| 143 | 2741 | ATAAGGCCTGACATGTGGCA |
| 144 | 2761 | TGGAATTACTCACCCTTAGC |
| 145 | 2781 | CCTGCATATCTAGTGCACCA |
| 146 | 2800 | GCUCCTTGAGATTAGCAGCC |
| 147 | 2821 | TTTATTCCCTCTGCACTGGA |
| 148 | 2841 | CTCTGTATTTTAGTCTAGCA |
| 149 | 2861 | TGAAATGCCCACCTGGAAGA |
| 150 | 2881 | GGTTCCTTGACTGAGTTGGT |
| 151 | 2901 | AAATGACTTTCTTTGTCATG |
| 152 | 2921 | ACTTGATGAGGTAAGAGTTG |
| 153 | 2941 | GTTTTCTCTGTCTTTATTCA |
| 154 | 2961 | AAAGAACAATGTCTCTTTCT |
| 155 | 2981 | TCCATTCAAAAGACTCAGGA |
| 156 | 3001 | GGCTATAACATAATACAATT |
| 157 | 3021 | TACCAAAATGGTTTTATGAT |
| 158 | 3041 | ACACCCAGTTCAGTCAAAAC |
| 159 | 3061 | AATCAAAAAGGAAAAAGTGA |
| 160 | 3081 | GTAGAATTTAAATTGTATTC |
| 161 | 3101 | TTGACGACTGCAGTCATCAA |
| 162 | 3121 | CATCTTGCATCAGGAGCCCC |
| 163 | 3141 | CAGACTTAAAATGGAAGGGG |
| 164 | 3161 | CTTTAACCTCTGTAAGGAGA |
| 165 | 3181 | CTTAGGAATTAGCCACTAGA |
| 166 | 3201 | GCATGTGTTAATCAGGTTTC |
| 167 | 3221 | AATGACCAGGATGGTTGTGA |
| 168 | 3241 | AAAAATAGAACATGCTCGAG |
| 169 | 3261 | ATATCAGGGGTGATTAGTTA |
| 170 | 3281 | GGATATATAAAAATAAAAAT |
| 171 | 3301 | GACGTAAAAAAATGAAAACT |
| 172 | 3321 | TGATATTAGCTTATAGGCAA |
| 173 | 3341 | GTCTTAAACAACCTTATTTA |
| 174 | 3361 | AATATGGATATTTGAAGCAC |
| 175 | 3381 | TTCCTTGAAAAATAGTGGTT |
| 176 | 3401 | CAGAGTGTACTTTTCCATAC |
| 177 | 3421 | GACATCGAGTGACAAAGTGA |
| 178 | 3441 | GTAGGCAATAACTTTGGAAT |
| 179 | 3461 | TTCATGACAGTCATTACTTA |
| 180 | 3481 | CAAATTATTTCAATGCTGCT |
| 181 | 3501 | AAAAGAGTGCCCCCTTTAAA |
| 182 | 3521 | CGGAAATTTTCTTCCCGTTT |
| 183 | 3541 | TCCATGATAAGACCAGGAAG |
| 184 | 3568 | CUUCCTTCCTGCCTCTAGCC |
| 185 | 3581 | TGAGGTCATCCCACTTCCTT |
| 186 | 3601 | ATCAAGAAAAGGTGACCTCC |
| 187 | 3621 | TCAGCCCATATGTTTCTGGA |
| 188 | 3641 | ATGAGGTCACCCCGGGTTTA |
| 189 | 3661 | CTTCTGCTGCAACTCATTTC |
| 190 | 3681 | CTTGTTCTGAAAAAAATAAA |
| 191 | 3701 | CAGAGGTCCATCAAACATCA |
| 192 | 3721 | TGTGTCTCCCTAAAGAGATT |
| 193 | 3741 | GGGGAGGGATCCCAGCCATC |
| 194 | 3761 | CTGGCAGTGAGAAGGGTACA |
| 195 | 3781 | TACCTTCACACGTAGTTCTC |
| 196 | 3801 | TGTATACTCCCTGCCTTGAA |
| 197 | 3821 | TGCCCAACAGGAAACAGCAA |
| 198 | 3841 | AAAATGTGGTCAAGGAGCAT |
| 199 | 3861 | TGATAACATCCACTCTTCCC |
| 200 | 3881 | CAGACACATTGTTTTCTCAA |
| 201 | 3901 | TATAAGAACCCCATTAATTC |
| 202 | 3921 | TCTTTTCTGGGAACCTTCTT |
| 203 | 3941 | TGAGGAGGCTGGATGAACAT |
| 204 | 3961 | TTTCTTGAATGTTCTGTTTC |
| 205 | 3981 | GATGACATCCTGATTGTCCT |
| 206 | 4001 | GTTTTTATTTTCATTTCCCT |
| 207 | 4021 | TAAGGTGATATCTCATTGTG |
| 208 | 4041 | AGTAGCCATTCTACCTGGTA |
| 209 | 4061 | ATGACACTTCATTTTTTTAT |
| 210 | 4081 | CCAATTTCTCTATATCCTTG |
| 211 | 4101 | TCCAGCAGTGAAGAAGGGTT |
| 212 | 4121 | CTACACCATTTTCCATTCCC |
| 213 | 4141 | CGTACTGTTTTTCATAACGG |
| 214 | 4161 | TTTAATTTTTGAGAAACCTC |
| 215 | 4181 | GATCATATAGCAGTTCTATT |
| 216 | 4201 | ATACAGAAGTGAGATTGCTG |
| 217 | 4221 | TTCAATTATTTTGGGTATAT |
| 218 | 4241 | TATTTTCTTGAAATTCTGAT |
| 219 | 4261 | AATGAACATGGGAGTGTAAA |
| 220 | 4281 | GTGATTGTGAAGAGTGCCAC |
| 221 | 4301 | TTTCCATAACTTTGGAAACA |
| 222 | 4321 | TTCAATGGAAATTTGGGTTG |
| 223 | 4341 | TTTTCTTTGTCCATTTATTT |
| 224 | 4361 | CCATTGTACGTATATGCACA |
| 225 | 4381 | TTTTTAGGCTGAATAATATC |
| 226 | 4401 | AAATAACAGGATTCCCCCTT |
| 227 | 4421 | GGGTTTATTCATGTTGTCAT |
| 228 | 4441 | TACATAGCATAATGGCCTCC |
| 229 | 4461 | TTTCTGTTACTTGCTCATTT |
| 230 | 4481 | GAAATCAGGCAGTATTTGTC |
| 231 | 4501 | TTTTAGAACCTCATATAAAT |
| 232 | 4521 | TGCTTCTATGAGTTTGACTA |
| 233 | 4541 | GGAACCACTGTTCTATTCTC |
| 234 | 4561 | TCCCTTCCTCCTTTTCCCTA |
| 235 | 4581 | ACTCCCTATTTCCTCATTTC |
| 236 | 4601 | AATTTTATACCAATTAGACA |
| 237 | 4621 | TAATTCATCTTGCATACTAT |
| 238 | 4641 | ATACAGCTGATCTTTAGAGC |
| 239 | 4661 | TTCATTATACGAACTCTGCT |
| 240 | 4681 | AAGTGCATAATACAGTATTG |
| 241 | 4701 | TACCCTCTTAACAAAATGTT |
| 242 | 4721 | AGAACACTTAACATGAGAGG |
| 243 | 4741 | TTGTGTATATGTATATGGTA |
| 244 | 4761 | CATCACCTCCAAAAGCTTCC |
| 245 | 4781 | ATCAAGGTAATAAATATATC |
| 246 | 4801 | CCTGTCAAACCATCACCACA |
| 247 | 4821 | AGTTTAGACATAGTCACATA |
| 248 | 4841 | TTAATGTATACAATTTGATG |
| 249 | 4861 | TATTATAAAACTGCATATAT |
| 250 | 4881 | CTTCATTCAGACATAATTGA |
| 251 | 4901 | TTGTCTTTTCTTTTTTATAG |
| 252 | 4921 | GTTTTGACAACTGAATTTTG |
| 253 | 4941 | TGACTGTGGTCATATTTCCA |
| 254 | 4961 | CACTCAGTAACAAACACTTC |
| 255 | 4981 | AAACCAAACACACTCTGAAA |
| 256 | 4990 | GACCTGCTCAAACCAAACAC |
| 257 | 5001 | CAATCACCCTAGACCTGCTC |
| 258 | 5021 | GAAACACACCCAGGGATGTT |
| 259 | 5041 | TCACTAGTACATGAGACATG |
| 260 | 5061 | ACAAATGCACACATCTACTT |
| 261 | 5081 | GGATACATAGGGATATGTGC |
| 262 | 5101 | AATACACACAGCCCTGATAG |
| 263 | 5121 | TGCGGACACACACACTTTCA |
| 264 | 5141 | CTTCTATACAGATATGATCA |
| 265 | 5161 | AAGAAATATAATCACACTCT |
| 266 | 5181 | TTCAAATGGATGTATTCTTC |
| 267 | 5201 | AAACAGCCATAGACATCCAT |
| 268 | 5221 | AAGAGTAGAGAACTCATCTC |
| 269 | 5241 | GGAGACTACTGTACAAGCAC |
| 270 | 5261 | ACCAAGCATAAGGGATAAGG |
| 271 | 5281 | TGGGGTCTAAGAACGTATCC |
| 272 | 5301 | TGCGGTCTCAGAGATCCACT |
| 273 | 5321 | ATATGAGGTTTGGTACCATC |
| 274 | 5341 | TATAGGAAAAAATATTGCAT |
| 275 | 5361 | CTTTATCTTAGGTATTTATG |
| 276 | 5381 | TGCCTAATTCAGAAGATGAA |
| 277 | 5401 | TTATTGTTAATCTCTTACTG |
| 278 | 5421 | CTATTCAATTTTATTGTTAG |
| 279 | 5441 | ATTACAATATATTATTATAA |
| 280 | 5461 | ATCACATTCACATAACTTTT |
| 281 | 5481 | TGAGAGAGAGAAAGAAAGAG |

AS is an abbreviation for antisense. Underlined nucleotides are 2′-β-methylribonucleotides; all others are 2′-deoxyribonucleotides. In the exemplary antisense oligonucleotides according to the invention, when a “CG” dinucleotide is contained in the sequence, such oligonucleotide is modified to remove or prevent the immune stimulatory properties of the oligonucleotide.

In another aspect, the invention provides a composition comprising at least one optimized antisense oligonucleotide according to the invention and a physiologically acceptable carrier, diluent or excipient. The characteristics of the carrier will depend on the route of administration. Such a composition may contain, in addition to the synthetic oligonucleotide and carrier, diluents, fillers, salts, buffers, stabilizers, solubilizers, and other materials well known in the art. The pharmaceutical composition of the invention may also contain other active factors and/or agents which enhance inhibition of TLR4 expression. For example, combinations of synthetic oligonucleotides, each of which is directed to different regions of the TLR4 mRNA, may be used in the pharmaceutical compositions of the invention. The pharmaceutical composition of the invention may further contain nucleotide analogs such as azidothymidine, dideoxycytidine, dideoxyinosine, and the like. Such additional factors and/or agents may be included in the pharmaceutical composition to produce a synergistic, additive or enhanced effect with the synthetic oligonucleotide of the invention, or to minimize side-effects caused by the synthetic oligonucleotide of the invention. The pharmaceutical composition of the invention may be in the form of a liposome in which the synthetic oligonucleotides of the invention is combined, in addition to other pharmaceutically acceptable carriers, with amphipathic agents such as lipids which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of such liposomal formulations is within the level of skill in the art, as disclosed, for example, in U.S. Pat. Nos. 4,235,871; 4,501,728; 4,837,028; and 4,737,323. The pharmaceutical composition of the invention may further include compounds such as cyclodextrins and the like that enhance delivery of oligonucleotides into cells or slow release polymers.

In another aspect, the invention provides a method of inhibiting TLR4 expression. In this method, an oligonucleotide or multiple oligonucleotides of the invention are specifically contacted or hybridized with TLR4 mRNA either in vitro or in a cell.

In another aspect, the invention provides methods for inhibiting the expression of TLR4 in a mammal, particularly a human, such methods comprising administering to the mammal a compound or composition according to the invention. One skilled in the art would recognize that the antisense compounds and compositions according to the invention can be administered through a variety of means. One such means for administration is according to Example 3. The antisense activity of a compound or composition according to the invention can be determined by measuring TLR4 mRNA and TLR4 protein concentration. The data is anticipated to demonstrate that administration of an exemplary TLR4 antisense oligonucleotide according to the invention can cause down-regulation of TLR4 expression in vivo.

In another aspect, the invention provides a method for inhibiting a TLR-mediated immune response in a mammal, the method comprising administering to the mammal a TLR4 antisense oligonucleotide according to the invention in a pharmaceutically effective amount, wherein routes of administration include, but are not limited to, parenteral, intramuscular, subcutaneous, intraperitoneal, intraveneous, mucosal delivery, oral, sublingual, transdermal, topical, inhalation, intranasal, aerosol, intraocular, intratracheal, intrarectal, vaginal, by gene gun, dermal patch or in eye drop or mouthwash form. One skilled in the art would recognize that one such administration can be accomplished according to Example 3, or by known methods. The antisense activity of compound or composition according to the invention can be determined by measuring biomarkers related to TLR4 signaling, for example, but not limited to, measuring IL-12. The data is anticipated to demonstrate that administration of an exemplary TLR4 antisense oligonucleotide according to the invention can cause down-regulation of TLR4 expression in vivo and prevent the induction of IL-12 by a TLR4 agonist. More generally, the data is anticipated to demonstrate the ability of a TLR4 antisense oligonucleotide according to the invention to inhibit the induction of pro-inflammatory cytokines by a TLR4 agonist.

In another aspect, the invention provides a method for therapeutically treating a mammal having a disease mediated by TLR4, such method comprising administering to the mammal, particularly a human, a TLR4 antisense oligonucleotide of the invention in a pharmaceutically effective amount.

In certain embodiments, the disease is cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a disease caused by a pathogen. Preferred autoimmune disorders include without limitation lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. In certain embodiments, inflammatory disorders include without limitation airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

In another aspect, the invention provides methods for preventing a disease or disorder in a mammal, particularly a human, at risk of contracting or developing a disease or disorder mediated by TLR4. Such method comprises administering to the mammal a prophylactically effective amount of an antisense oligonucleotide or composition according to the invention. Such diseases and disorders include, without limitation, cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a disease caused by a pathogen in a vertebrate. Autoimmune disorders include, without limitation, lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. Inflammatory disorders include, without limitation, airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

In another aspect, the invention provides a method for inhibiting TLR4 expression and activity in a mammal, comprising administering to the mammal an antisense oligonucleotide complementary to TLR4 mRNA and an antagonist of TLR4 protein, a kinase inhibitor or an inhibitor of STAT protein. Accordingly, TLR4 expression is inhibited by the antisense oligonucleotide, while any TLR4 protein residually expressed is inhibited by the antagonist. Preferred antagonists include anti-TLR4 antibodies or binding fragments or peptidomimetics thereof, RNA-based compounds, oligonucleotide-based compounds, and small molecule inhibitors of TLR4 activity or of a signaling protein's activity.

In the various methods according to the invention, a therapeutically or prophylactically effective amount of a synthetic oligonucleotide of the invention and effective in inhibiting the expression of TLR4 is administered to a cell. This cell may be part of a cell culture, a neovascularized tissue culture, or may be part or the whole body of a mammal such as a human or other mammal. Administration of the therapeutic compositions of TLR4 antisense oligonucleotide can be carried out using known procedures at dosages and for periods of time effective to reduce symptoms or surrogate markers of the disease, depending on the condition and response, as determined by those with skill in the art. It may be desirable to administer simultaneously, or sequentially a therapeutically effective amount of one or more of the therapeutic TLR4 antisense oligonucleotides of the invention to an individual as a single treatment episode. In some exemplary embodiments of the methods of the invention described above, the oligonucleotide is administered locally and/or systemically. The term “administered locally” refers to delivery to a defined area or region of the body, while the term “systemic administration” is meant to encompass delivery to the whole organism.

In any of the methods according to the invention, one or more of the TLR4 antisense oligonucleotide can be administered alone or in combination with any other agent useful for treating the disease or condition that does not diminish the immune modulatory effect of the TLR4 antisense oligonucleotide. In any of the methods according to the invention, the agent useful for treating the disease or condition includes, but is not limited to, one or more vaccines, antigens, antibodies, cytotoxic agents, allergens, antibiotics, antisense oligonucleotides, TLR agonists, TLR antagonists, siRNA, miRNA, aptamers, peptides, proteins, gene therapy vectors, DNA vaccines, adjuvants or kinase inhibitors to enhance the specificity or magnitude of the immune response, or co-stimulatory molecules such as cytokines, chemokines, protein ligands, trans-activating factors, peptides and peptides comprising modified amino acids. For example, in the treatment of autoimmune disease, it is contemplated that the TLR4 antisense oligonucleotide may be administered in combination with one or more targeted therapeutic agents and/or monoclonal antibodies. Alternatively, the agent can include DNA vectors encoding for antigen or allergen. In these embodiments, the TLR4 antisense oligonucleotide of the invention can produce direct immune modulatory or suppressive effects. When co-administered with one or more other therapies, the synthetic oligonucleotide of the invention may be administered either simultaneously with the other treatment(s), or sequentially.

In the various methods according to the invention the route of administration may be, without limitation, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% synthetic oligonucleotide and preferably from about 25 to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution or glycols such as ethylene glycol, propylene glycol or polyethylene glycol. When administered in liquid form, the pharmaceutical composition contains from about 0.5 to 90% by weight of the synthetic oligonucleotide or from about 1 to 50% synthetic oligonucleotide.

When a therapeutically effective amount of synthetic oligonucleotide of the invention is administered by parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form, the synthetic antisense oligonucleotide will be in the form of a pyrogen-free, parenterally acceptable aqueous solution. The preparation of such parenterally acceptable solutions, having due regard to pH, isotonicity, stability, and the like, is within the skill in the art. A pharmaceutical composition for parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection or other vehicle as known in the art. The pharmaceutical composition of the present invention may also contain stabilizers, preservatives, buffers, antioxidants or other additives known to those of skill in the art.

When administered parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form, doses ranging from 0.01% to 10% (weight/volume) may be used. When administered in liquid form, a liquid carrier such as water, petroleum, oils of animal or plant origin such as peanut oil, mineral oil, soybean oil, sesame oil or synthetic oils may be added. Topical administration may be by liposome or transdermal time-release patch.

The amount of synthetic oligonucleotide in the pharmaceutical composition of the present invention will depend upon the nature and severity of the condition being treated, and on the nature of prior treatments which the patient has undergone. It is contemplated that the various pharmaceutical compositions used to practice the method of the present invention should contain about 10 micrograms to about 20 mg of synthetic oligonucleotide per kg body or organ weight.

The duration of intravenous therapy using the pharmaceutical composition of the present invention will vary, depending on the severity of the disease being treated and the condition and potential idiosyncratic response of each individual patient.

Some diseases lend themselves to acute treatment while others require longer term therapy. Both acute and long term intervention in diseases are worthy goals. Injections of antisense oligonucleotides against TLR4 can be an effective means of inhibiting certain diseases in an acute situation. However for long term therapy over a period of weeks, months or years, systemic delivery (intraperitoneal, intramuscular, subcutaneous, intravenous) either with carriers such as saline, slow release polymers or liposomes are likely to be considered.

In some chronic diseases, systemic administration of oligonucleotides may be preferable. The frequency of injections is from continuous infusion to once a month, several times per month or less frequently will be determined based on the disease process and the biological half life of the oligonucleotides.

The oligonucleotides and methods of the invention are also useful for examining the function of the TLR4 gene in a cell or in a control mammal or in a mammal afflicted with a disease associated with TLR4 or immune stimulation through TLR4. In such use, the cell or mammal is administered the oligonucleotide, and the expression of TLR4 mRNA or protein is examined.

Without intending to be limited to any theory or mechanism, it is generally believed that the activity of oligonucleotides according to the invention depends on the hybridization of the oligonucleotide to the target nucleic acid (e.g. to at least a portion of a genomic region, gene or mRNA transcript thereof), thus disrupting the function of the target. Such hybridization under physiological conditions is measured as a practical matter by observing interference with the function of the nucleic acid sequence. Thus, an exemplary oligonucleotide used in accordance with the invention is capable of forming a stable duplex (or triplex in the Hoogsteen or other hydrogen bond pairing mechanism) with the target nucleic acid; activating RNase H or other in vivo enzymes thereby causing effective destruction of the target RNA molecule; and is capable of resisting nucleolytic degradation (e.g. endonuclease and exonuclease activity) in vivo. A number of the modifications to oligonucleotides described above and others which are known in the art specifically and successfully address each of these exemplary characteristics.

The patents and publications cited herein reflect the level of knowledge in the art and are hereby incorporated by reference in their entirety. Any conflict between the teachings of these patents and publications and this specification shall be resolved in favor of the latter. Those skilled in the art will recognize, or be able to ascertain, using no more than routine experimentation, numerous equivalents to the specific substances and procedures described herein. For example, antisense oligonucleotides that overlap with the oligonucleotides may be used. Such equivalents are considered to be within the scope of this invention.

The following examples illustrate the exemplary modes of making and practicing the present invention, but are not meant to limit the scope of the invention since alternative methods may be utilized to obtain similar results.

### EXAMPLES

### Example 1

### Preparation of TLR4-Specific Antisense Oligonucleotides

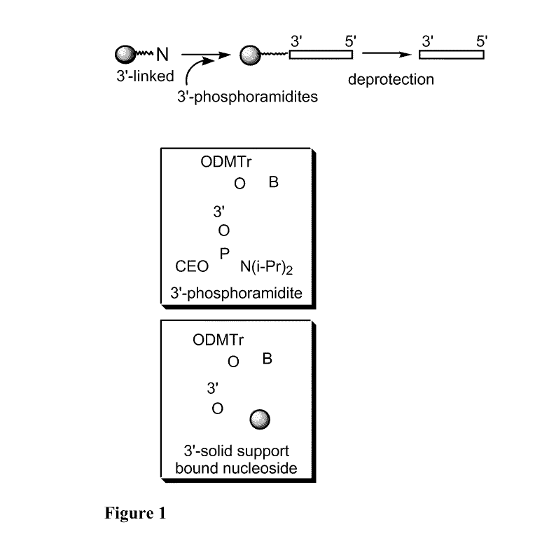

Chemical entities according to the invention were synthesized on a 1 μmol to 0.1 mM scale using an automated DNA synthesizer (OligoPilot II, AKTA, (Amersham) and/or Expedite 8909 (Applied Biosystem)), following the linear synthesis procedure outlined in FIG. 1.

5′-DMT dA, dG, dC and T phosphoramidites were purchased from Proligo (Boulder, Colo.). 5′-DMT 7-deaza-dG and araG phosphoramidites were obtained from Chemgenes (Wilmington, Mass.). DiDMT-glycerol linker solid support was obtained from Chemgenes. 1-(2′-deoxy-β-D-ribofuranosyl)-2-oxo-7-deaza-8-methyl-purine amidite was obtained from Glen Research (Sterling, Va.), 2′-O-methylribonuncleoside amidites were obtained from Promega (Obispo, Calif.). All compounds according to the invention were phosphorothioate backbone modified.

All nucleoside phosphoramidites were characterized by ³¹P and ¹H NMR spectra. Modified nucleosides were incorporated at specific sites using normal coupling cycles recommended by the supplier. After synthesis, compounds were deprotected using concentrated ammonium hydroxide and purified by reverse phase HPLC, detritylation, followed by dialysis. Purified compounds as sodium salt form were lyophilized prior to use. Purity was tested by CGE and MALDI-TOF MS. Endotoxin levels were determined by LAL test and were below 1.0 EU/mg.

### Example 2

### Cell Culture Conditions and Reagents

### HEK293 Cell Culture Assays for TLR4 Antisense Activity

HEK293 cells stably expressing human TLR4/CD14/MD-2 (Invivogen, San Diego, Calif.) were plated in 48-well plates in 250 μL/well DMEM supplemented with 10% heat-inactivated FBS in a 5% CO₂ incubator. At 80% confluence, cultures were transiently transfected with 400 ng/mL of the secreted form of human embryonic alkaline phosphatase (SEAP) reporter plasmid (pNifty2-Seap) (Invivogen) in the presence of 4 μL/mL of lipofectamine (Invitrogen, Carlsbad, Calif.) in culture medium. The SEAP reporter plasmid is inducible by NF-κB. Plasmid DNA and lipofectamine were diluted separately in serum-free medium and incubated at room temperature for 5 min. After incubation, the diluted DNA and lipofectamine were mixed and the mixtures were incubated further at room temperature for 20 min. Aliquots of 25 μL of the DNA/lipofectamine mixture containing 100 ng of plasmid DNA and 1 μL of lipofectamine were added to each well of the cell culture plate, and the cells were transfected for 6 h. After transfection, medium was replaced with fresh culture medium (no antibiotics), antisense compounds were added to the wells, and incubation continued for 18-20 h. Cells were then stimulated with the human TLR4 agonist, LPS, at 12.5 ng/ml for 6 h.

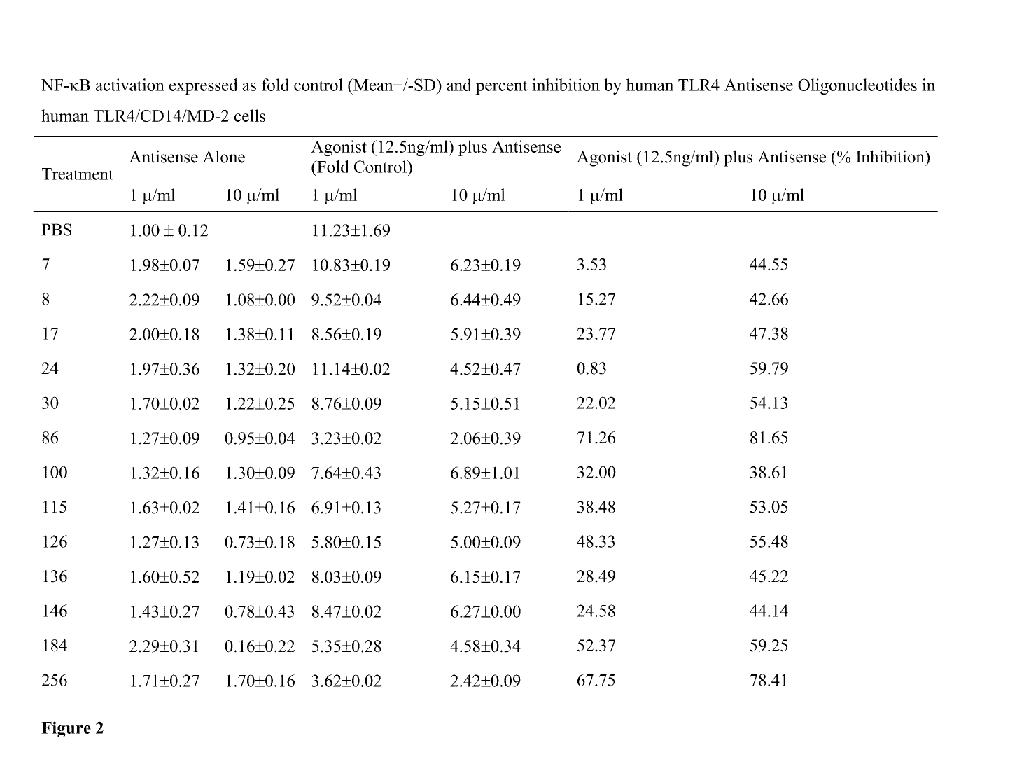

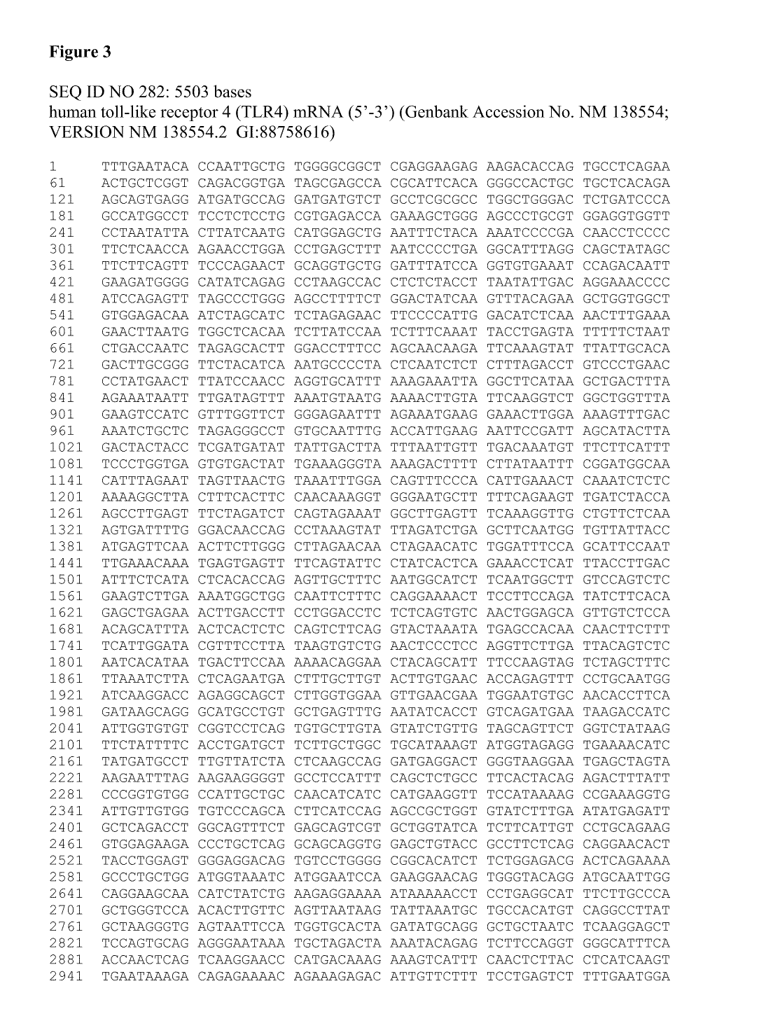

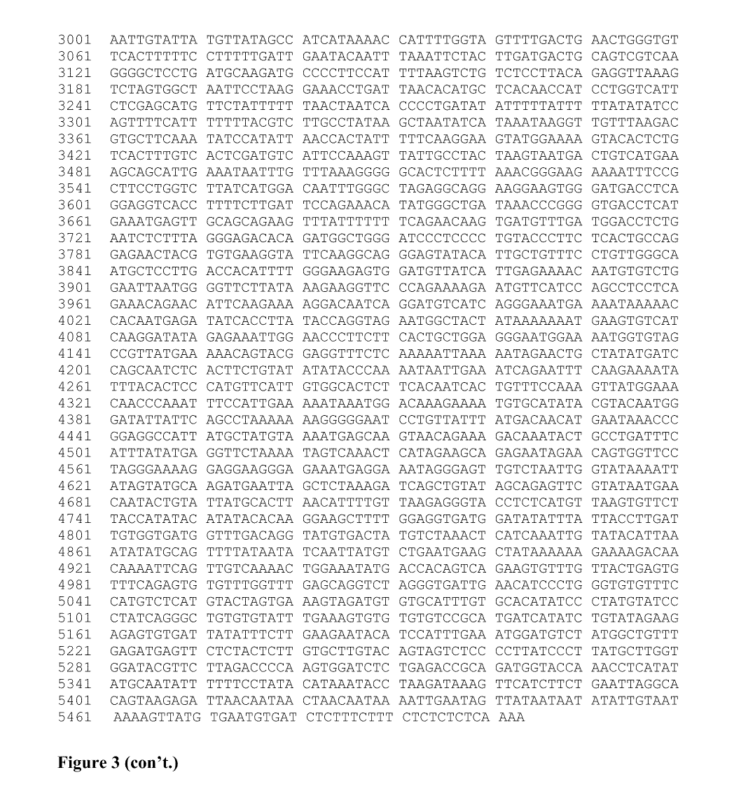

At the end of the treatment, 20 μL of culture supernatant was taken from each well and assayed for SEAP by the Quanti Blue method according to the manufacturer's protocol (Invivogen). The data are depicted in FIG. 2. The data in FIG. 2 depict NF-κB activity compared to control and demonstrate (i) that exemplary human TLR4 antisense oligonucleotides according to the invention are not immunostimulatory (Antisense Alone); and (ii) that exemplary human TLR4 antisense oligonucleotides according to the invention inhibit TLR4 expression and activation (Agonist plus Antisense).

### Example 3

### In Vivo Activity of TLR4 Antisense Oligonucleotide

Female C57BL/6 mice of 5-6 weeks age (N=3/group) are injected with exemplary murine TLR4 antisense oligonucleotides according to the invention at 5 mg/kg, or PBS, subcutaneously once a day for three days. Subsequent to administration of the TLR4 antisense oligonucleotide, mice are injected with 0.25 mg/kg of a TLR4 agonist subcutaneously. Two hours after administration of the TLR4 agonist, blood is collected and TLR4 mRNA, TLR4 protein, and IL-12 concentrations are determined by ELISA.

What is claimed is:

1. A synthetic antisense oligonucleotide 20 to 50 nucleotides in length complementary to TLR4 mRNA (SEQ ID NO: 282), wherein the antisense oligonucleotide has a sequence comprising SEQ ID NOs: 7, 8, 17, 24, 30, 49, 86, 100, 102, 115, 121, 126, 136, 146, 184 or 256, and wherein the oligonucleotide specifically hybridizes to and inhibits the expression of human TLR4.

2. A composition comprising a synthetic antisense oligonucleotide according to claim 1 and a physiologically acceptable carrier.

3. A method for inhibiting the expression of TLR4, the method comprising administering a synthetic antisense oligonucleotide according to claim 1.

4. A method for inhibiting the expression of TLR4, the method comprising administering a composition according to claim 2.

5. A method for inhibiting the expression of TLR4 in a mammal, the method comprising administering to the mammal a synthetic antisense oligonucleotide according to claim 1.

6. A method for inhibiting the expression of TLR4 in a mammal, the method comprising administering to the mammal a composition according to claim 2.

7. A method for inhibiting a TLR4-mediated immune response in a mammal, the method comprising administering to the mammal a synthetic antisense oligonucleotide according to claim 1 in a pharmaceutically effective amount.

8. A method for inhibiting a TLR4-mediated immune response in a mammal, the method comprising administering to the mammal a composition according to claim 2 in a pharmaceutically effective amount.

9. A method for therapeutically treating a mammal having one or more diseases or disorders mediated by TLR4, the method comprising administering to the mammal a synthetic antisense oligonucleotide according to claim 1 in a pharmaceutically effective amount.

10. A method for therapeutically treating a mammal having one or more diseases or disorders mediated by TLR4, the method comprising administering to the mammal a composition according to claim 2 in a pharmaceutically effective amount.

11. A method for preventing in a mammal one or more diseases or disorders mediated by TLR4, the method comprising administering to the mammal a synthetic antisense oligonucleotide according to claim 1 in a prophylactically effective amount.

12. A method for preventing in a mammal one or more diseases or disorders mediated by TLR4, the method comprising administering to the mammal a composition according to claim 2 in a prophylactically effective amount.

13. A method for down-regulating TLR4 expression and thus preventing undesired TLR4-mediated immune stimulation by a compound that activates TLR4, the method comprising administering a synthetic antisense oligonucleotide according to claim 1 in combination with one or more compounds that would activate a TLR4-mediated immune response but for the presence the antisense oligonucleotide.

14. A method for down-regulating TLR4 expression and thus preventing undesired TLR4-mediated immune stimulation by a compound that activates TLR4, the method comprising administering a composition according to claim 2 in combination with one or more compounds that would activate a TLR4-mediated immune response but for the presence of the composition.

15. The method according claim 5, wherein the mammal is a human.

16. The method according to claim 9, wherein the one or more diseases or disorders are selected from the group consisting of cancer, an autoimmune disease or disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma and a disease caused by a pathogen.

17. The method according to claim 16, wherein the autoimmune disease or disorder is selected from the group consisting of lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis.

18. The method according to claim 16, wherein the inflammatory disease or disorder is selected from the group consisting of airway inflammation, asthma, autoimmune diseases or disorders, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

19. The method according to claim 3, wherein the route of administration is selected from the group consisting of parenteral, intramuscular, subcutaneous, intraperitoneal, intraveneous, mucosal delivery, oral, sublingual, transdermal, topical, inhalation, intranasal, aerosol, intraocular, intratracheal, intrarectal, vaginal, gene gun, dermal patch, eye drop and mouthwash.

20. The method according to claim 3, comprising further administering one or more vaccines, antigens, antibodies, cytotoxic agents, allergens, antibiotics, antisense oligonucleotides, TLR agonists, TLR antagonists, siRNA, miRNA, kinase inhibitors, aptamers, proteins, gene therapy vectors, DNA vaccines, adjuvants, co-stimulatory molecules or combinations thereof.

21. A method for inhibiting TLR4 expression and activity in a mammal, comprising administering to the mammal an antisense oligonucleotide complementary to TLR4 mRNA and an antagonist of TLR4 protein.

22. The method according to claim 21, wherein the TLR4 antagonist is selected from the group consisting of anti-TLR antibodies or binding fragments or peptidomimetics thereof, RNA-based compounds, oligonucleotide-based compounds, and small molecule inhibitors of TLR4 activity.

23. The method according to claim 11, wherein the one or more diseases or disorders are selected from the group consisting of cancer, an autoimmune diseases or disorder, airway inflammation, inflammatory diseases or disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma and a disease caused by a pathogen.

24. The method according to claim 23, wherein the autoimmune disease or disorder is selected from a group consisting of lupus erythematosus, multiple sclerosis, type I diabetes mellitus, irritable bowel syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis.

25. The method according to claim 21, wherein the inflammatory disease or disorder is selected from a group consisting of airway inflammation, asthma, autoimmune diseases or diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.
